(19) United States
(12) Patent Application Publication
(10) Pub. No.: US 2025/0283847 A1
(43) Pub. Date: Sep. 11, 2025

(54) MICRONEEDLE-BASED PLANT SENSORS AND METHODS OF MAKING AND USING THEREOF

(71) Applicant: BOARD OF REGENTS, THE UNIVERSITY OF TEXAS SYSTEM, Austin, TX (US)

(72) Inventor: Shawana TABASSUM, Tyler, TX (US)

(73) Assignee: BOARD OF REGENTS, THE UNIVERSITY OF TEXAS SYSTEM, Austin, TX (US)

(21) Appl. No.: 18/859,101

(22) PCT Filed: Apr. 27, 2023
(86) PCT No.: PCT/US2023/020228

§ 371 Date: Oct. 22, 2024

Related U.S. Application Data
(60) Provisional application No. 63/336,409, filed on Apr. 29, 2022.

(51) Int. Cl. G01N 27/403; G01N 27/27; G01N 27/327; G01N 33/00

(52) U.S. Cl. CPC G01N 27/403; G01N 27/27; G01N 27/3275; G01N 33/0098

(57) ABSTRACT

A plant sensor comprising a plurality of microneedles that is capable of continuous in situ measurement without the use of bioagents and a method of using the plant sensor. The plant sensor includes a biocompatible polymer substrate and one or more sensors disposed on the substrate includes a plurality of microneedles and electrodes. The plant sensor includes real time measurement and pH-correction capabilities.

### CROSS-REFERENCE TO RELATED APPLICATION

This application claims priority benefit of U.S. Provisional Application No. U.S. 63/336,409, filed Apr. 29, 2022, which is incorporated herein by reference in its entirety.

### STATEMENT OF GOVERNMENTAL SUPPORT

Part of the work performed during development of this invention utilized U.S. Government funding support under Award Number ECCS-2138701 issued by the National Science Foundation. The U.S. Government has certain rights in this invention.

### TECHNICAL FIELD

The present disclosure relates to a multi-parametric plant sensor comprising a plurality of microneedles. The plant sensor is capable of continuous in situ measurement without the use of bioagents.

### BACKGROUND

With the world population expected to rise to 9.8 billion by 2050, there is a proportional increase in the food demand. Therefore, optimizing the use of agrochemicals and reducing crop productivity losses are essential to cope with the growing need for food worldwide. Plants are subjected to various abiotic stressors, including drought and salinity, and biotic stressors, including pest and herbivore attacks. Studies report that drought stress alone yielded 21% and 40% of global reductions in wheat and maize productions, respectively, between 1980 and 2015. Plants have sophisticated systems to detect and respond to environmental stimuli. Plants respond and adapt to these environmental stresses by phytohormone-mediated regulation of oxidative stresses. Research shows correlations between the stressors and phytohormone levels in plants. The impacts of these stressors on crop yield are often not detected early enough to mitigate crop damage. Environmental stresses induce a progressive change in the levels of phytohormones, which are circulated throughout the plant via xylem and phloem. Thus, the levels of phytohormones can serve as early signals of plant stress. Real-time measurement of plant parameters is crucial to gauge the adverse ecological impacts and intervene early.

Salicylic acid (SA), jasmonic acid (JA), abscisic acid (ABA), and indole-3-acetic acid (IAA) are among the most important regulators of induced defense mechanisms. Progressive variations in their levels have been reported in many drought, salt, and cold/heat-stressed plants. Moreover, exogenous application of these hormones mitigates oxidative stress in plants. The interconnected signaling pathways of these hormones are central to the plant's ability to fine-tune the induction of defenses in response to stresses. However, the dynamic interaction mechanism of these hormones under environmental stress conditions is not fully elucidated due to the lack of knowledge and technology needed to facilitate the use of in situ sensors.

The postharvest qualities of fruits and vegetables depend not only on postharvest management practices but also on preharvest monitoring and treatment. Fruits and vegetables that are inappropriately maintained before harvest, can never be improved in quality by any postharvest treatment. Therefore, it is imperative to investigate and control the preharvest factors that are directly associated with the quality of the harvest. Monitoring the progression of plant growth and ripening is crucial to obtain high quality produce and determine the time to harvest. Ripening is a complex process governed by a myriad of factors including hormonal balance. Although the critical role of hormones in plant development has been well established, previous research was mostly centered around how singular hormones affect ripening. For instance, an upsurge in ethylene production is observed in climacteric fruits such as tomatoes and bananas, while a progressive accumulation of the phytohormone abscisic acid is reported in non-climacteric fruits/vegetables such as grapes and bell peppers. However, there is a complex network of hormonal balance and their crosstalk regulates the ripening process. Endogenous salicylic acid (SA) and indole-3-acetic acid (IAA) play multiple roles in plant development and ripening. For instance, their levels are generally higher during the initial phases of development and subside progressively at later stages.

There have been no reports of integrated sensors that can provide adequate real-time hormonal measurements in situ. The conventional approaches to conducting molecular analyses of plants include liquid chromatography (LC), nuclear magnetic resonance (NMR) imaging, and infrared (IR) spectroscopy. Although LC and NMR methods are highly sensitive and selective, they are limited to laboratory settings. In addition, they are non-continuous, disruptive, time-consuming, laborious, and expensive (>$100 k). The tissue samples need to be collected in the field and brought to the laboratory periodically and throughout the growing season. Moreover, the collected samples lose their functionality due to the need to transport long distances to the lab. The time lag between sample collection and analysis prevents follow-up and dynamical studies. Due to the complexities and time delays associated with these techniques, only a few plants are sampled, and extrapolations are made about the whole population, thereby neglecting the significant variations between plants across a field. The infrared and thermal imaging techniques provide a non-disruptive view of the action of the stressors in plants. Still, they lack accuracy, do not provide quantitative analysis of metabolites, and are effective only at late crop responses.

Some existing sensors are either glass/ceramic substrate-based and hence are not compliant with plants or are flexible substrate-based but require human intervention for sample collection. Some require a hole to be punched into the plants. Thus, measured phytohormone levels may be inaccurate as a result of such variability using existing sensors that lack pH correction capabilities.

Additionally, sap pH levels vary with crop growth and ambient stressors. Variations in the sap pH indicate the state of plant health including risk potential for insect damage, foliage disease attack, nutritional imbalance, and stress levels. Environmental stresses cause a progressive change in the levels of secondary metabolites circulating in plants. Some key stress-associated metabolites include salicylic acid, abscisic acid, and amino acids, which are acidic in nature. These compounds are found in the xylem/phloem sap and their concentrations change in response to environmental stress conditions, resulting in a corresponding change in the sap pH level.

Conventional leaf patches lack sap monitoring and rely on ligand chemical bonding, limiting their durability and lifespan. Additionally, they lack system-level integration and wireless data communication, thereby limiting cloud storage or end user applications.

Unmanned aerial systems have emerged as an attractive tool for aerial scouting. They can fly to waypoints, hover, and collect high-resolution data (millimeters per pixel) from large acres of the field quickly. However, they do not conduct chemical profiling of the plant and are very power-hungry, requiring human intervention for battery recharge/replacement. It can take an entire 8-hour workday to exhaustively collect high-definition images from every zone in an 80-acre crop field. The commercial in-situ crop sensors (e.g., FloraPulse and Dynamax) do not provide real-time and continuous monitoring of stress responses in plants. They only monitor sap water content and do not provide chemical measurements (e.g., metabolite or nutrient), hence stress profiling.

Other methods to assess the quality and maturity of fruits and vegetables include infrared spectroscopy, imaging and machine vision, and electronic noses. Although these methods provide non-destructive and multiplexed analysis of several internal attributes of the fruit/vegetable, they are discrete, bulky, manually operated, lack spatiotemporal information, and often effective at later stages of ripening.

Thus, none of the previously reported plant monitoring technologies can provide both real-time and continuous assessment of stress responses in plants. There is an unmet need for plant sensors that can provide continuous and in situ monitoring capabilities.

### SUMMARY

The present disclosure provides real-time and in situ monitoring of plants that can differentiate the effect of individual stressors and their combinations on individual plant productivity during its various growth stages and reduce the loss of crops. In some aspects, the present disclosure provides both real-time and continuous assessment of stress responses in plants, multiplexed detection of stress-related hormones (simultaneous detection of multiple phytohormones), wireless data transfer capability, and an energy efficient and low-cost solution, while incurring minimal damage to the plant. The inventor has surprisingly found that a plant sensor comprising a plurality of microneedles provides continuous and highly accurate in situ measurement of one or more physiological and/or environmental parameters of a plant. Real-time continuous crop profiling will help growers implement timely and site-specific applications of agrochemicals to mitigate potential impacts in production.

The present disclosure is directed to a plant sensor, comprising a biocompatible polymer substrate; and one or more sensors disposed on the substrate comprising a plurality of microneedles. In some aspects the microneedles have a height dimension of 100 μm to 5,000 μm and a base-width dimension of 100 μm to 5,000 μm. In some aspects, the microneedles have a vertex angle of 10° to 50°. In certain aspects, the microneedles have a bending angle of less than 15° at a pressure of 600 kPa, or a bending angle of 100 or less at a pressure of 200 kPa, or a bending angle of 3° or less at a pressure of 5 kPa.

In some aspects, the plant sensor comprises at least one sensor configured to measure a physical parameter and/or at least one sensor configured to measure a chemical parameter. In certain aspects, the sensors are each independently configured to detect humidity, temperature, pH, multiplexed phytohormones, or volatile organic compounds.

In some aspects, the present invention includes a plant sensor and further comprises a data acquisition system in communication with one or more sensors. In some aspects, the data acquisition system includes a processor; a communication unit; and a power supply unit. In some aspects, the data acquisition system includes a microprocessor; a communication unit; and a power supply unit. In some aspects, the data acquisition system includes a microcontroller; a communication unit; and a power supply unit. A plant sensor according to any of the foregoing aspects, may further include a voltage booster. In some embodiments, the data acquisition system further comprises a voltage booster. The voltage booster may be connected to two or more electrodes. In certain embodiments, the data acquisition system may include one or more processors and memory, which may be coupled together with a bus. The one or more processors and other components may be coupled together with a bus, a separate bus, or may be directly connected together or coupled using a combination of the foregoing. The memory may contain executable code or software instructions that when executed by the one or more processors or processing circuitry cause the one or more processors or processing circuitry to perform the techniques disclosed herein. The memory may be configured to store the one or more calibration plots and/or other instructions.

The data acquisition system is configured according to the parameters being measured by the plant sensor. The present invention also includes a kit comprising the plant sensor including any of the additional components and/or features described herein. In some aspects, the kit may include the sensor, the data acquisition unit, and/or a user manual or instructions. In some aspects, the user manual or instructions may provide instructions and explanations for sensor installation and data acquisition capabilities and uses of the plant sensor. In some aspects, a kit may, instead of a user manual, provide a hyperlink or quick response (QR) code leading to an internet site or mobile application hosting instructions and explanations for sensor installation and data acquisition capabilities and uses of the plant sensor.

The present invention is also directed to a plant sensor, comprising a biocompatible polymeric substrate; and two or more electrodes or preferably three or more electrodes disposed on the substrate. Each electrode may comprise a plurality of coated microneedles. The plant sensor according to the present disclosure advantageously prevents potential drift. For example, potential drifts caused by large currents passing through the two electrodes are prevented using three or more electrodes as described herein. Thus, the inventor has found that the three-electrode setup is able to provide superior electrochemical analysis. In some aspects, the microneedles have a bending angle of less than 150 at a pressure of 600 kPa.

In some embodiments, the sensor is configured to detect one or more conditions, including, but not limited to, pH, temperature, water content, or humidity and/or one or more analytes, including, but not limited to phytohormones, phytochemicals, or volatile organic compounds. In some embodiments, the sensor is configured to detect concentrations of Salicylic acid (SA), jasmonic acid (JA), abscisic acid (ABA), and indole-3-acetic acid (IAA). In some embodiments, the sensor is configured to detect a pH in a range of about 1 to 14, or pH in a range of about 2 to 13.

In some aspects, the sensor is configured to detect a phytohormone concentration of at least 0.10 μM, or at least 0.25 μM, or at least 0.5 μM, or at least 1 μM, or at least 25 μM, or at least 37 μM. In some aspects, the detection range may include phytohormone concentrations from 1 μM and 0.10 μM. In some aspects, the detection range is up to 1000 μM.

In some aspects, the sensor is configured to detect a phytohormone with a deviation of less than 10%, or less than 5%, or less than 1%, or less than 0.5%, or less than 0.1%, wherein the deviation is across at least three repeated measurements.

In some aspects, the microneedles may have a height dimension of 200 μm to 4,000 μm, or 300 μm to 3,000 μm, or 400 μm to 2,000 μm, or 500 μm to 1,000 μm, or 600 μm to 800 μm and the microneedles may have a base-width dimension of 200 μm to 4,000 μm, or 300 μm to 3,000 μm, or 400 μm to 2,000 μm, or 500 μm to 1,000 μm, or 600 μm to 800 μm. In a preferred embodiment, the microneedles have a height dimension of 800 μm and a base-width dimension of 800 μm. In other preferred aspects, the microneedles have a height dimension of 2000 μm and a base-width dimension of 800 μm.

In some aspects, plant sensor may have a width of 0.25 cm to 1.5 cm, or 0.5 cm to 1 cm, or 0.75 cm, and a length of 0.75 cm to 2.5 cm, or 1 cm to 2 cm, or 1.5 cm. The width and length of the plant sensor may be adjusted as needed according to the plant stem and/or leaf size or such as to allow a plurality of plant sensors to be placed on a single plant.

The plant sensor of the present disclosure has advantageously minimal footprint and effects on the plant.

In some aspects, the plant sensor has a surface area of about 1 to 15 cm², or 3 to 12 cm², or 4 to 8 cm², or 5 to 7 cm².

In some aspects, the plant sensor is 50 grams or less, or 25 grams or less, or 10 grams or less, or 5 grams or less. In preferred aspects, the plant sensor is about 5 grams.

In some aspects, the microneedles may have a vertex angle of 20° to 50°, or 30° to 40°. In preferred aspects, the vertex angle is 30°. In some aspects, the plant sensor may further comprise a power supply unit and an electrode control unit. The power supply unit and the electrode control unit are in communication with and in operative control of the three or more electrodes. In certain aspects, the electrode control unit comprises a potentiostat and/or a data acquisition system. The data acquisition system may include additional onboard sensors, including e.g., temperature or humidity. In some aspects, the onboard sensors may provide resistance variations in response to measured parameters, such as temperature or humidity. In some aspects, the data acquisition system may include a microprocessor and/or a microcontroller with a built-in analog to digital converter. In some aspects, the power supply unit that comprises a battery. In some aspects, the battery can run the flexible plant sensor for at least 120 days. In some aspects, the battery may be a 3.6V battery.

In some aspects, the plant sensor may include a reference electrode (RE), a counter electrode (CE), and at least one working electrode (WE). The electrodes may be configured according to a certain microneedle coating. In some aspects, the microneedle coating is selected from a graphene ink, an Ag/AgCl paste, a metal organic framework (MOF), a graphene hydrogel nanocomposite, a poly(3,4-ethylenedioxythiophene) polystyrene sulfonate (PEDOT:PSS) cross-linked with 3-glycidyloxypropyl)trimethoxysilane (GOPS), a polyaniline (PANI) based nanofiber, or a combination thereof. In some aspects, the metal organic framework comprises at least one metal selected from copper, zinc, or gold. In some aspects, the graphene hydrogel nanocomposite is a gold nanoparticle decorated graphene hydrogel nanocomposite (AuNP-GH). In some aspects, the least one WE is coated with graphene ink.

In certain aspects, the least one WE is coated with graphene ink and an additional coating selected from the group consisting of a metal organic framework (MOF), a gold nanoparticle decorated graphene hydrogel nanocomposite (AuNP-GH), a poly(3,4-ethylenedioxythiophene) polystyrene sulfonate (PEDOT:PSS) cross-linked with 3-glycidyloxypropyl)trimethoxysilane (GOPS), and a polyaniline (PANI) based nanofiber. In certain aspects, the CE is coated with graphene ink. In some aspects, the RE is coated with Ag/AgCl paste.

In some aspects, the plant sensor comprises a communication unit and an electrode control unit that includes a non-transitory computer-readable medium, such as memory storage, communicatively coupled to a processor, the non-transitory computer-readable medium having stored thereon computer software comprising a set of instructions that, when executed by the processor, causes the electrode control unit to receive electrode data from each of the three or more electrodes; and send, via the communication unit, the sensor data to an external device. In some aspects, the electrode data from each of the three or more electrodes is sent, via the communication unit, to an Internet of Things (IoT) cloud server configured to interact/communicate with one or more IoT-capable devices. In some aspects, the electrode data from each of the three or more electrodes is sent, via the communication unit, to an external device. In some aspects, the data are sent via a wired connection. In some aspects, the data are sent via a wireless connection. In some embodiments, the communication module implements a communication protocol based on Bluetooth or Bluetooth low energy transmission, Wi-Fi, Wi-Max, IEEE 802.11 technology, a radio frequency (RF) communication. In some embodiments, the communication module implements a communication protocol based on general packet radio service (GPRS), enhanced data GSM environment (EDGE), long term evolution-advanced (LTE-A), LTE, 3G, 4G, 5G, code division multiple access (CDMA), wideband CDMA (WCDMA), evolution-data optimized (EVDO), wireless broadband Internet (Wibro), Mobile WiMax, Digital Enhanced Cordless Telecommunications (DECT), Digital AMPS (IS-136/TDMA, Integrated Digital Enhance Network (iDEN), HSPA+, Flash-OFDM, HIPERMAN, WiFi, IBurst, UMTS, W-CDMA, HSPDA+HSUPA, UMTS-TDD and other formats for utilizing cell phone technology, telephony antenna distributions and/or any combinations thereof, and including the use of satellite, microwave technology, the internet, cell tower, telephony and/or public switched telephone network lines. In some embodiments, the communication module implements a communication protocol based on near field communication (NFC).

In some aspects the communication unit is part of the electrode control unit and/or the data acquisition system. In some embodiments, the communication unit is a separate unit from the electrode control unit and/or the data acquisition system.

In some aspects, the instructions are configured to select one or more calibration plots to analyze at least one electrode data. In certain aspects, the instructions are configured to perform a signal calibration of at least one electrode. The calibration may comprise at least one of a pH-based signal correction, a temperature-based signal correction, or a humidity-based signal correction.

In certain aspects, the plant sensor of the present disclosure comprises onboard pH- and temperature-correction features.

The present invention is also directed to a method for continuously measuring one or more phytohormones in a plant, comprising attached to the plant: (i) a reference electrode (RE); (ii) a counter electrode (CE); and (iii) one or more working electrodes (WE) configured to detect a phytohormone wherein each electrode comprises a plurality of microneedles, and wherein each electrode is operatively connected to an electrode control unit. The method further comprises applying a potential corresponding to a peak current for the one or more phytohormones; measuring at least one signal correction parameter; and determining the concentration of the one or more phytohormones based on the peak current using a pre-determined calibration plot, wherein the pre-determined calibration plot is based on the value of the measured correction parameter.

In some aspects, the signal correction parameter comprises temperature, humidity, pH, or an analyte. The analyte may be a second phytohormone.

In certain aspects, the electrode control unit may be at least one of a potentiostat and a data acquisition system.

In some aspects, the method includes a continuous measurement for at least 120 days, or at least 90 days, or least 60 days, or at least 30 days, or at least two weeks, or at least 10 days, or at least 7 days. In some aspects, the measurement may be conducted over a period of 5 to 30 seconds, 30 to 60 seconds, 1-30 μminutes, for example 1 μminute to 4 μminutes, 1-24 hours, or may be conducted over 24, 36, 48, 60, 72, or more hours. Measurements may be continuously taken for 1, 2, 3, 4, 5, or 6 weeks, or 1, 2, 3, 4, 5, or 6 μmonths. Measurements may be continuously taken for part or all of a growing season.

The sensors according to any of the embodiments described herein may be able to detect peak currents with high stability for long periods of time. In some embodiments, the sensors may show a decrease in peak current detection of less than 10%, 9%, 8%, 7%, 6%, 5%, 4%, 3%, 2%, or 1%, including any values in between the foregoing percentages. In some embodiments, the above stability is maintained over at least one day, at least one week, two or more weeks, at least one month, one or more months, or over an entire growing season. In some embodiments, a peak current value detected by any of the sensors described herein may have a decrease of 2.5% or less or 1.5% or less over at least seven days. In some embodiments, SA, IAA, and ET sensors are able to achieve a peak current value showing a decrease of 2.5% or less or 1.5% or less over at least seven days.

The sensors according to any of the embodiments described herein are highly selective for target analytes relative to interfering species. For example, the sensors according to any of the embodiments described herein will detect a higher signal for target analytes relative to a signal detected from interfering species alone. In some embodiments, the selectivity of the sensors according to any of the embodiments described herein may be at least 50× higher for target analytes relative to a signal corresponding to interfering species. In some embodiments, the selectivity for target analytes is at least 1.1×, 1.2×, 1.3×, 1.4×, 1.5×, 2×, 3×, 4×, 5×, 6×, 7×, 8×, 9×, 10×, 11×, 12×, 13×, 14×, 15×, 16×, 17×, 18×, 19×, 20×, 25×, 30×, 40×, or 50× higher than a signal for one or more interfering species. The selectivity, i.e., signal, for target analytes may be 1.1× to 50× relative to one or more interfering species. In some embodiments, the selectivity for target analytes is any value within the foregoing ranges.

In some aspects, the method includes sending electrode data, via the communication unit, to an Internet of Things (IoT) cloud server configured to interact/communicate with one or more IoT-capable devices. In some aspects, the method includes sending electrode data, via the communication unit, to an external device via a wired connection. In some aspects, the method includes sending electrode data to an external device wirelessly. In some aspects, the external device and/or IoT-capable device may include, but is not limited to, a desktop computer, a laptop computer, typical cell phone, smart device (e.g., smart phones), or similar apparatus including all remote cellular phones using channel access methods defined above (with cellular equipment, public switched telephone network lines, satellite, tower and mesh technology), mobile phones, PDAs, tablets (e.g. refers to all current and future variants, revisions and generations of the Apple WAD, Samsung Galaxy, HP, Acer, Microsoft, Nook, Google Nexus, Sony, Kindle and all future tablets manufactured by these and other manufactures), Apple IPOD Touch, or a television, watch, timepiece or fob watch and other similar apparatus with WIFI and wireless capability, and controllers having internet or wireless connectivity.

In some aspects, the method includes selecting one or more calibration plots to analyze the electrode data and/or performing a signal calibration. In some embodiments, the one or more calibration plots may be stored on an external device and the data acquisition system may be configured to cause the external device to transmit the one or more calibration plots to the data acquisition system via the cloud server.

The calibration may include at least one of a pH-based signal correction, a temperature-based signal correction, a humidity-based signal correction, or a signal calibration based on the signal of an analyte. A microneedle-based electrochemical sensor for in situ monitoring of SA/JA/ABA/IAA in live plants is not reported in the literature. Additionally, in a field condition, sap pH levels vary with crop growth and ambient stressors. The plant sensor of the present disclosure is the first of its kind to provide a microneedle-based electrochemical sensor for in situ monitoring of SA in live plants with correction of SA measurements.

In some aspects, the electrodes are attached to the plant leaf and/or stem. In some aspects, the electrodes are attached to at least two locations of the same plant. The method according to the present invention may include measuring kinetics of the one or more phytohormones in the plant and/or the distribution of the one or more phytohormones in the plant. In some aspects, the method further comprises continuously measuring one or more phytohormones on a plurality of plants.

It will be readily appreciated that the methods provided in this disclosure may be used to modify and/or augment various crop growing procedures. In certain aspects, the method further comprises modifying an irrigation control system in response to the concentration of the one or more phytohormones, modifying and/or applying a pesticide treatment in response to the concentration of the one or more phytohormones, and/or modifying and/or applying a fertilizer or nutrient treatment in response to the concentration of the one or more phytohormones. In some aspects, the method comprises harvesting the plant or a plurality of plants in response to the concentration of the one or more phytohormones.

In certain aspects, the method further comprises continuously measuring a second parameter selected from a physical parameter or a chemical parameter in response to the concentration of the one or more phytohormones. The second parameter may comprise humidity, temperature, soil conditions, plant growth, additional phytohormones, or volatile organic compounds.

According to the method of the present disclosure, the one or more phytohormones are detected with a deviation of less than 10%, or less than 5%, or less than 1%, or less than 0.5%, or less than 0.1%, wherein the deviation is across at least three repeated measurements.

### BRIEF DESCRIPTION OF DRAWINGS

FIG. 1A shows a schematic illustration of a plant sensor of the present disclosure (left) and a single microneedle of the illustrative plant sensor (right). FIGS. 1B to 1C show microscopic images (top and side views) of a microneedle sensor of the present disclosure.

FIG. 2A shows a plot of a CuMOF coating using Fourier transform infrared spectroscopy (FTIR). FIG. 2B shows a plot of an AuNP-GH using UV-Vis spectroscopy.

FIG. 3A shows DPV responses for an SA electrode and FIG. 3B shows a calibration plot of the SA electrode sensor for varying SA concentrations (sensitivity=0.005 μM⁻¹). FIG. 3C shows DPV responses for IAA and FIG. 3D shows calibration plot of the TAA electrode sensor for varying IAA concentrations (sensitivity=0.76149 μA M⁻¹).

FIGS. 4A-4B show selectivity relative to common interfering species found in sap for the SA electrode sensor FIG. 4A and the IAA electrode sensor FIG. 4B.

FIGS. 5A-5B show concentration response curves for four repeated measurements of SA FIG. 5A and IAA in FIG. 5B. FIGS. 5C-5D show SA and IAA calibration plots for temperature variations ranging from 10° C. to 55° C. FIGS. 5E-5F show cyclic tests for hormone levels of SA in FIG. 5E and IAA in FIG. 5F.

FIG. 6A shows an image of the plant sensor mounted on a leaf FIG. 6B shows calibration plot of the temperature sensor achieving a sensitivity of 0.0886 kΩ/° C. FIG. 6C shows Real-time SA variations in water-stressed vs. control plants for 7 days. FIG. 6D shows SA dynamics over 12 hr, repeated with 3 plants.

FIGS. 7A-7C show step by step fabrication of the electrodes. The 3D printed structure shown in FIG. 7A with the working electrodes and the counter electrode coated with graphene ink and subsequently cured in FIG. 7B. The reference electrode is coated with Ag/AgCl paste and subsequently cured in FIG. 7C. An optical image of the microneedle structure is shown in FIG. 7D (scale bar=0.5 cm).

FIG. 8A shows cyclic voltammetry (CV) responses for PANI deposition on the WEpH (50 cycles). FIG. 8B shows calibration curve of the pH sensor (the error bars represent three repeated measurements).

FIG. 9A shows differential Pulse Voltammetry (DPV) responses for different concentrations of SA. FIG. 9B shows Calibration curve showing I_{SA}/I_{CuMOF} vs. SA concentrations (measurements were repeated 3 times).

FIG. 10 shows selectivity relative to common interfering species found in sap for the SA electrode sensor.

FIG. 11 shows calibration curves of the SA sensor for different sap pH (4.09, 7.1, and 10.14).

FIG. 12 shows an experimental setup for real-time SA measurements on the stem of a cabbage plant including a potentiostat and data acquisition system (DAS).

FIG. 13A shows SA measurement results on the stem of unstressed and water-stressed cabbage plants. FIG. 13B shows SA measurement results at two different locations on the same plant. FIG. 13C shows pH measurement results on the stem of unstressed and water-stressed cabbage plants. FIG. 13D shows pH measurement results at two different locations on the same plant. Error bars represent 3 repeated measurements.

FIG. 14 shows stress-strain characteristics of the microneedles.

FIG. 15 shows a reduced-in-scale schematic for a plant sensor system.

FIG. 16 shows a flow diagram of the process for real-time monitoring of a condition in a plant.

FIG. 17 shows an exemplary configuration of a plant sensor system.

FIGS. 18A-18B shows a calibration curve depicting the measured voltage as a function of sap pH levels from a microneedle sensor patch (FIG. 18A) and a reproducibility test conducted with three identical pH sensors (FIG. 18B). Error bars show 3 repeated measurements.

FIG. 19 shows measured changes in pH within the sap of a plant in response to salinity stress over time using three concentrations of NaCl. Concentrations of NaCl are indicated in the graph.

FIGS. 20A-20C show the fabrication process for an electrode suite. FIG. 20A shows the 3D printed microneedle electrodes. An exemplary screen printing process for an ethylene sensor is depicted in FIG. 20B. FIG. 20C depicts a sensor suite attached to a plant and interfaced with a drone.

FIGS. 21A-21D show plots for electrochemical measurements using differential pulse voltammetry (DPV) for SA and IAA. Differential Pulse Voltammetry (DPV) responses for different concentrations of SA are shown in FIG. 21A. A SA calibration curve showing I_{SA}/I_{CuWOF} vs. SA concentrations is shown in FIG. 21B. DPV responses for different concentrations of IAA are shown in FIG. 21C. An IAA calibration curve showing I_{IAA} vs. IAA concentrations is shown in FIG. 21D.

FIGS. 22A-22D show plots for Cyclic Voltammetry (CV) measurements used to conduct electrochemical characterization of an ethylene sensor. CV responses for different concentrations of ethylene are shown in FIG. 22A. An ethylene calibration curve showing current vs. ethylene concentrations are shown in FIG. 22B. CV responses for PANI deposition on an electrode is shown in FIG. 22C. A plot for a pH sensor calibration curve is shown in FIG. 22D. Error bars represent 3 repeated measurements.

FIGS. 23A-23C show plots for a selectivity test for SA (FIG. 23A) and IAA (FIG. 23B) sensors. A plot for Selectivity test for ethylene sensor is shown in FIG. 23C.

FIGS. 24A-24B show calibration curves of SA (FIG. 24A) and IAA (FIG. 24B) sensors for different pH conditions.

FIGS. 25A-25D show trends of SA and IAA levels in unripe (FIG. 25A) and ripe (FIG. 25B) bell peppers. FIG. 25C shows trends of ethylene in ripe and unripe bell peppers. FIG. 25D shows a plot of stability for SA, IAA, and ET sensors over one week.

### DETAILED DESCRIPTION

The terms “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example, ±20%, ±10%, or ±5% may be within the intended meaning of the recited value where appropriate. Numerical quantities given are approximate, meaning that the term “around,” “about” or “approximately” can be inferred if not expressly stated.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

A “plant sensor” means a device configured to attach to a plant and to sense one or more conditions, including, but not limited to, pH, temperature, water content, or humidity and/or one or more analytes, including, but not limited to phytohormones, phytochemicals, or volatile organic compounds. In some embodiments, the plant sensor may be a plant patch or a leaf patch, such as a microneedle patch, configured to attach to a part of a plant, such as a plant stem or one or more leaves.

A “plant sensor system” means one or more components in addition to the plant sensor, including, but not limited to, a potentiostat, one or more of the processors, a communication unit, a power supply unit, and/or a data acquisition system. A plant sensor system may include additional components to perform any of the techniques described herein.

A “plant sensor suite” means a sensor configuration having a plurality of sensors. In some embodiments a plant sensor suite includes different types of sensors. In some embodiments, the different types of sensors may include sensors for measuring different analytes. In some embodiments, the different sensors may include different sensor configurations, including, but not limited to, one or more microneedle based sensors or sensor arrays and one or more screen printed (or drop cast) sensors.

A “processor” means one or more microprocessors, central processing units (CPUs), processing circuitry, computing devices, one or more microcontrollers, digital signal processors, or like devices or any combination thereof, regardless of the architecture (e.g., chip-level multiprocessing/multi-core, RISC, CISC, Microprocessor without Interlocked Pipeline Stages, pipelining configuration, simultaneous multithreading). In some embodiments, the processor is operatively connected to memory. The processor and memory may be connected externally or internally.

“Biocompatible polymer” means any synthetic (man made) or natural polymers which are suitable to be used in the close vicinity of a living system or work in intimacy with living tissue. Examples of biocompatible polymers include, but are not limited to, polyethylenes, polyvinyl chlorides, polyarnides, such as nylons, polyesters, rayons, polypropylenes, polyacrylonittiles, auylics, polyisoprenes, polybufadienes and polybutadiene-polyisoprene copolymers, neoprenes and nitrile rubbers, polyisobutylenes, olefinic rubbers, such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates, such as ethylene vinyl acetate copolymer, homopolymers and copolymers of acrylates, such as polymethylmethacrylate, polyethylmethacrylate, polyrnethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers, such as polytetrafluoroethylene and polyvinyl fluoride, polystyrenes, homopolymers and copolymers of styrene acrylonitrile, cellulose acetates, homopolymers and copolymers of acrylonitrile butadiene styrene, polyrnethylpentenes, polysulfones, polyesters, polyimides, polyisobutylenes, polymethylstyrenes, and other similar compounds known to those skilled in the art.

In some embodiments, the biocompatible polymer comprises a photopolymer resin. In some embodiments, the biocompatible polymer comprises a mixture of methacrylic acid esters and a photoinitiator.

“Computer-readable medium” means any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include random access memory (RAM) or dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, SecureDigital (SD™) memory card, USB Flash Drives, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

A “drone” means a device that is autonomous or unmanned, such that it does not have a human operator onboard or require continuous instructions from a human operator. A drone may include, but is not limited to, an unmanned aerial vehicle (UAV), unmanned ground vehicle (UGV), or an unmanned stationary device. The drone may include an interface for receiving, stowing, providing one or more items, or connecting and/or communicating by means of data or information transfer to designated items, for example one or more sensors. The interface may include active and/or passive mechanisms that couple to an item and secure the item with the drone during transportation in flight or on the ground. In addition, the mechanisms may decouple the item from the drone upon reaching a designated destination, e.g., to provide the item at a designated location, to pick up an item at a designated location. In an embodiment, the mechanism includes an actuated mechanical arm with a grip interface to attach and de-attach the item. The drone may include any number of sensors for data collection, navigation, landing, or other functionality. Additionally, the drone may include one or more motors (e.g., electric motors) for actuating one or more rotors, wheels, tracks, or other means of travel. In some embodiments, the drone may include more than one motor. An onboard battery, which may be rechargeable, provides power for the motors as well as other functionality of the drone. The drone may be configured to operate remotely without a wired connection or via a wired connection.

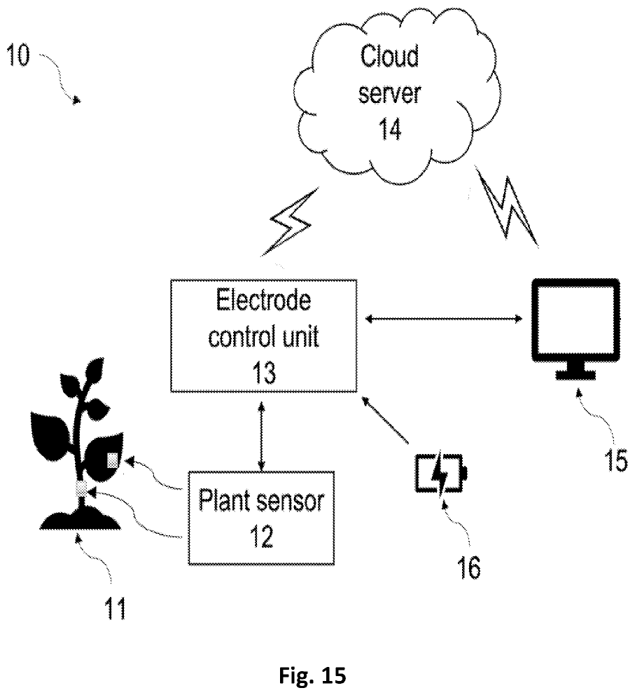

FIG. 15 shows a reduced-in-scale schematic for a plant sensor system 10, which comprises one or more plant sensors 12 attached to a plant 11 in one or multiple locations on the plant. The plant sensor 12 communicates with an electrode control unit 13 connected to a power supply 16. The electrode control unit is configured to receive data from plant sensor 12 and send the sensor data to a cloud server 14 via the communication unit (not shown). In some embodiments, the communication unit is a separate unit from the electrode control unit. The cloud server 14 may be a IoT cloud server configured to interact/communicate with one or more external devices 15, e.g., an IoT-capable device. The sensor data may be sent from the cloud server 14 to an external device 15. In some embodiments, data from the electrode control unit 13 is sent via a wired connection to an external device 15. It will be appreciated that other configurations of these elements are possible.

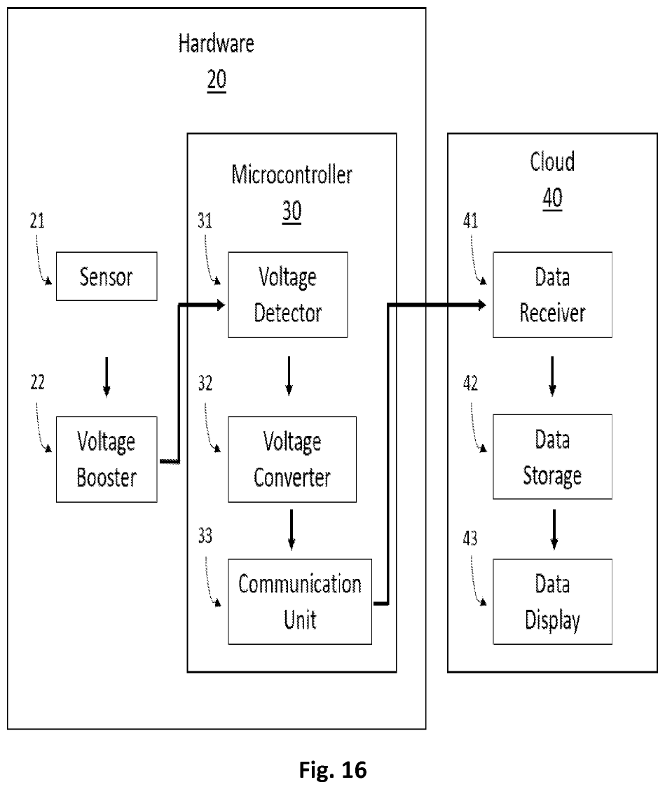

FIG. 16 shows a flow diagram of the process for real-time monitoring of pH in the leaf of a plant according to certain embodiments. Plant sensor hardware 20 include a sensor 21 for attachment to a plant part connected to a voltage booster 22 to enhance the voltage signal. The microcontroller 30 processes and analyzes the signal via the voltage detector 31 and the voltage converter 32. In some embodiments, an analog voltage may be converted to a digital signal by an in-built analog to digital converter. The voltage converter may be configured to convert voltage to one or more plant condition parameters. In some embodiments, voltage is converted based on a predetermined calibration curve. Voltage can be converted to one or more conditions, including, but not limited to, pH, temperature, water content, or humidity and/or one or more analytes, including, but not limited to phytohormones, phytochemicals, or volatile organic compounds. The data is transferred via a communication unit 33 (such as a Wifi data transmitter) to a cloud system 40 comprising a data receiver 41 to receive data to be stored in a data storage device 42, which can be displayed via one or more data display devices 43.

In some embodiments, the present disclosure includes any one or combination of the following non-limiting numbered items:
1. A plant sensor, comprising:
a biocompatible polymer substrate; and
one or more sensors disposed on the substrate, wherein each of the one or more sensors comprises a plurality of microneedles,
wherein:
a) the microneedles have a height dimension of 100 μm to 5,000 μm and a base-width dimension of 100 μm to 5,000 μm;
b) the microneedles have a vertex angle of 3° to 90°;
c) the microneedles have a bending angle of less than 150 at a pressure of 600 kPa;
d) the microneedles have a height dimension of 100 μm to 5,000 μm and a base-width dimension of 100 μm to 5,000 μm and the microneedles have a vertex angle of 3° to 90°;
e) the microneedles have a height dimension of 100 μm to 5,000 μm and a base-width dimension of 100 μm to 5,000 μm and the microneedles have a bending angle of less than 15° at a pressure of 600 kPa;
f) the microneedles have a vertex angle of 3° to 900 and the microneedles have a bending angle of less than 150 at a pressure of 600 kPa; or
g) the microneedles have a height dimension of 100 μm to 5,000 μm and a base-width dimension of 100 μm to 5,000 μm, the microneedles have a vertex angle of 3° to 90°, and the microneedles have a bending angle of less than 150 at a pressure of 600 kPa.
2. The plant sensor of item 1, wherein the microneedles have a bending angle of 100 or less at a pressure of 200 kPa.
3. The plant sensor of item 1, wherein the microneedles have a bending angle of 3° or less at a pressure of 5 kPa.
4. The plant sensor of item 1, wherein at least one sensor is configured to measure a physical parameter and/or at least one sensor is configured to measure a chemical parameter.
5. The plant sensor of item 1, wherein the sensors are each independently configured to detect humidity, temperature, stem and/or leaf growth, pH, one or more phytohormones, or one or more volatile organic compounds.
6. The plant sensor of item 5, wherein at least one sensor is configured to detect Salicylic acid (SA), jasmonic acid (JA), abscisic acid (ABA), or indole-3-acetic acid (IAA).
7. The plant sensor of item 5, wherein at least one sensor is configured to detect a pH in a range of about 1 to 14, or pH in a range of about 2 to 13.
8. The plant sensor of item 1, wherein the microneedles have a height dimension of 200 μm to 4,000 μm, or 300 μm to 3,000 μm, or 400 μm to 2,000 μm, or 500 μm to 1,000 μm, or 600 μm to 800 μm.
9. The plant sensor of item 1, wherein the microneedles have a base-width dimension of 200 μm to 4,000 μm, or 300 μm to 3,000 μm, or 400 μm to 2,000 μm, or 500 μm to 1,000 μm, or 600 μm to 800 μm.
10. The plant sensor of item 1, wherein the microneedles have a vertex angle of 20° to 50°, or 300 to 40°.
11. The plant sensor of item 1, having a width of 0.25 cm to 1.5 cm, or 0.5 cm to 1 cm, or 0.75 cm, and a length of 0.75 cm to 2.5 cm, or 1 cm to 2 cm, or 1.5 cm.
12. The plant sensor of item 1, wherein the plant sensor comprises a pH sensor integrated therein and is configured to perform pH correction of measured salicylic acid (SA) levels.
13. The plant sensor of any one of items 1-10, further comprising a data acquisition system, wherein the data acquisition system comprises a processor; a communication unit; and a power supply unit, and wherein the data acquisition system is in communication with the one or more sensors.
14. The plant sensor of items 1-10, further comprising a voltage booster.
15. A plant sensor, comprising:
a biocompatible polymeric substrate;
three or more electrodes disposed on the substrate, wherein each electrode comprises a plurality of microneedles, and
   wherein the microneedles have a bending angle of less than 150 at a pressure of 600 kPa.
16. The plant sensor of item 15, wherein the sensor is configured to detect a phytohormone concentration of at least 0.10 μM, or at least 0.25 μM, or at least 0.5 μM, or at least 1 μM, or at least 25 μM, or at least 30 μM, or at least 35 μM, or at least 37 μM.
17. The plant sensor of item 15, wherein the sensor is configured to detect a phytohormone with a deviation of less than 10%, or less than 5%, or less than 1%, or less than 0.5%, or less than 0.1%, wherein the deviation is across at least three repeated measurements.
18. The plant sensor of item 15, wherein the microneedles have a coating thereon, wherein the coating has a thickness of 0.25 μm to 5 μm, or 0.5 μm to 3 μm, or 0.75 μm to 2.5 μm, or 1 μm to 2.0 μm, or 1.25 μm to 1.5 μm.
19. The plant sensor of item 15, wherein the microneedles have a height dimension of 100 μm to 5,000 μm, or 200 μm to 4,000 μm, or 300 μm to 3,000 μm, or 400 μm to 2,000 μm, or 500 μm to 1,000 μm, or 600 μm to 800 μm.
20. The plant sensor of item 15, wherein the microneedles have a base-width dimension of 100 μm to 5,000 μm, or 200 μm to 4,000 μm, or 300 μm to 3,000 μm, or 400 μm to 2,000 μm, or 500 μm to 1,000 μm, or 600 μm to 800 μm.
21. The plant sensor of item 15, wherein the microneedles have a vertex angle of 3° to 90°, or 20° to 50°, or 300 to 40°.
22. The plant sensor of item 15, having a width of 0.1 cm to 2 cm, or 0.25 cm to 1.5 cm, or 0.5 cm to 1 cm, or 0.75 cm, and a length of 0.5 cm to 5 cm, or 0.75 cm to 2.5 cm, or 1 cm to 2 cm, or 1.5 cm.
23. The plant sensor of items 15-22, wherein the plant sensor further comprises:
a power supply unit; and
an electrode control unit;
wherein the power supply unit and the electrode control unit are in communication with the three or more electrodes.
24. The plant sensor of item 23, wherein the electrode control unit comprises a potentiostat.
25. The plant sensor of item 23, wherein the electrode control unit comprises a data acquisition system.
26. The plant sensor of item 23, further comprising a voltage booster.
27. The plant sensor of items 15-22, comprising a reference electrode (RE), a counter electrode (CE), and at least one working electrode (WE).
28. The plant sensor of items 15-22, wherein the microneedle coating is selected from a graphene ink, an Ag/AgCl paste, a metal organic framework (MOF), a graphene hydrogel nanocomposite, a poly(3,4-ethylenedioxythiophene) polystyrene sulfonate (PEDOT:PSS) cross-linked with 3-glycidyloxypropyl)trimethoxysilane (GOPS), a polyaniline (PANI) based nanofiber, or a combination thereof.
29. The plant sensor of item 28, wherein the metal organic framework comprises at least one metal selected from copper, zinc, or gold.
30. The plant sensor of item 28, wherein the graphene hydrogel nanocomposite is a gold nanoparticle decorated graphene hydrogel nanocomposite (AuNP-GH).
31. The plant sensor of item 27, wherein the least one WE is coated with graphene ink.
32. The plant sensor of item 27, wherein the least one WE is coated with graphene ink and an additional coating selected from the group consisting of a metal organic framework (MOF), a gold nanoparticle decorated graphene hydrogel nanocomposite (AuNP-GH), a poly(3,4-ethylenedioxythiophene) polystyrene sulfonate (PEDOT:PSS) cross-linked with 3-glycidyloxypropyl)trimethoxysilane (GOPS), and a polyaniline (PANI) based nanofiber.
33. The plant sensor of item 27, wherein the CE is coated with graphene ink.
34. The plant sensor of item 27, wherein the RE is coated with Ag/AgCl paste.
35. The plant sensor of item 23, wherein the electrode control unit comprises a non-transitory computer-readable medium communicatively coupled to a processor, the non-transitory computer-readable medium having stored thereon computer software comprising a set of instructions that, when executed by the processor, causes the electrode control unit to: receive electrode data from each of the three or more electrodes; and send, via the communication unit, the sensor data to an external device.
36. The plant sensor of item 35, wherein the electrode data from each of the three or more electrodes is sent, via the communication unit, to an Internet of Things (IoT) cloud server configured to interact with one or more IoT-capable devices.
37. The plant sensor of item 35, wherein the electrode data from each of the three or more electrodes are sent, via the communication unit, to an external device.
38. The plant sensor of item 35, wherein the instructions are configured to select one or more calibration plots to analyze at least one electrode data.
39. The plant sensor of item 35, wherein the instructions are configured to perform a signal calibration of at least one electrode.
40. The plant sensor of item 39, wherein the calibration comprises a pH-based signal correction, a temperature-based signal correction, a humidity-based signal correction, or a combination thereof.
41. A method for continuously measuring one or more phytohormones in a plant, comprising:
attaching to the plant:
   i) a reference electrode (RE);
   ii) a counter electrode (CE); and
   iii) one or more working electrode (WE) configured to detect a phytohormone,
wherein each electrode comprises a plurality of microneedles, and
wherein each electrode is operatively connected to an electrode control unit;
applying a potential corresponding to a peak current for the one or more phytohormones; measuring at least one signal correction parameter;
determining the concentration of the one or more phytohormones based on the peak current using a pre-determined calibration plot, wherein the pre-determined calibration plot is based on the measured value of the at least one signal correction parameter.
42. The method of item 41, wherein the phytohormone is selected from Salicylic acid (SA), jasmonic acid (JA), abscisic acid (ABA), or indole-3-acetic acid (IAA).
43. The plant sensor of item 41, wherein at least one electrode is configured to detect a pH in a range of about 1 to 14, or pH in a range of about 2 to 13.
44. The method of item 41, wherein the at least one signal correction parameter is selected from temperature, humidity, pH, and an analyte.
45. The method of item 44, wherein the analyte is a second phytohormone.
46. The method of item 41, wherein the electrode control unit comprises at least one of a potentiostat and a data acquisition system.
47. The method of items 41-46, wherein the continuous measurement occurs for at least 120 days, or at least 90 days, or least 60 days, or at least 30 days, or at least two weeks, or at least 10 days, or at least 7 days.
48. The method of items 41-46, wherein the electrode control unit comprises a processor and a communication unit.
49. The method of item 48, wherein the electrode data from the electrodes is sent, via the communication unit, to an Internet of Things (IoT) cloud server configured to interact with one or more IoT-capable devices.
50. The method of item 48, wherein the electrode data from the electrodes are sent, via the communication unit, to an external device.
51. The method of item 48, wherein the instructions are configured to select one or more calibration plots to analyze the electrode data.
52. The method of item 48, wherein the instructions are configured to perform a signal calibration.
53. The method of item 52, wherein the calibration comprises at least one of a pH-based signal correction, a temperature-based signal correction, a humidity-based signal correction, or a signal calibration based on the signal of an analyte.
54. The method of items 41-53, wherein the electrodes are attached to a leaf of the plant.
55. The method of items 41-54, wherein the electrodes are attached to a stem of the plant.
56. The method of items 41-55, wherein the electrodes are attached to at least two locations of the same plant.
57. The method of item 56, further comprising measuring kinetics of the one or more phytohormones in the plant.
58. The method of item 56, further comprising measuring the distribution the one or more phytohormones in the plant.
59. The method of items 41-58, further comprising continuously measuring one or more phytohormones on one plant or a plurality of plants.
60. The method of items 41-59, further comprising modifying an irrigation control system in response to the concentration of the one or more phytohormones.
61. The method of items 41-60, further comprising modifying and/or applying a pesticide treatment in response to the concentration of the one or more phytohormones.
62. The method of items 41-61, further comprising modifying and/or applying a fertilizer or nutrient treatment in response to the concentration of the one or more phytohormones.
63. The method of items 41-62, further comprising harvesting the plant in response to the concentration of the one or more phytohormones.
64. The method of items 41-63, further comprising continuously measuring a second parameter selected from a physical parameter or a chemical parameter in response to the concentration of the one or more phytohormones.
65. The method of item 64, wherein the second parameter comprises humidity, temperature, soil conditions, plant growth, or volatile organic compounds.
66. The method of items 41-65, wherein the one or more phytohormones are detected with a deviation of less than 10%, or less than 5%, or less than 1%, or less than 0.5%, or less than 0.1%, wherein the deviation is across at least three repeated measurements.
67. A kit comprising the plant sensor of any one of items 1-39, 65, and 66, a data acquisition unit, and/or a user's manual.
68 The plant sensor of item 13, further comprising a potentiostat in communication with the one or more sensors, and wherein the potentiostat is in communication with one or more of the processor, the communication unit, the power supply unit, and the data acquisition system.
69. A plant sensor, comprising:
a biocompatible polymeric substrate;
two or more electrodes disposed on the substrate, wherein each electrode comprises a plurality of microneedles;
a power supply unit;
an electrode control unit; and
a voltage booster connected to the two or more electrodes;
wherein the microneedles have a height dimension of 100 μm to 5,000 μm and a base-width dimension of 100 μm to 5,000 μm and the microneedles have a vertex angle of 3° to 90°; and
wherein the power supply unit and the electrode control unit are in communication with the two or more electrodes.
70. The plant sensor of items 69, comprising a reference electrode (RE), at least one working electrode (WE), and optionally a counter electrode (CE).
71. The plant sensor of item 69, wherein the plurality of microneedles are coated with a coating selected from a graphene ink, an Ag/AgCl paste, a metal organic framework (MOF), a graphene hydrogel nanocomposite, a poly(3,4-ethylenedioxythiophene) polystyrene sulfonate (PEDOT:PSS) cross-linked with 3-glycidyloxypropyl)trimethoxysilane (GOPS), a polyaniline (PANI) based nanofiber, or a combination thereof.
72. The plant sensor of item 69, wherein the RE is coated with Ag/AgCl paste.
73. The plant sensor of item 69, wherein the WE is coated with PANI.
74. The plant sensor of items 69-73, wherein the electrode control unit comprises a non-transitory computer-readable medium communicatively coupled to a processor, the non-transitory computer-readable medium having stored thereon computer software comprising a set of instructions that, when executed by the processor, causes the electrode control unit to:
receive electrode data from each of the two or more electrodes; and
send, via the communication unit, the sensor data to an external device.
75. The plant sensor of items 69-73, wherein the electrode control unit comprises a voltage detector and a voltage converter.
76. The plant sensor of item 74, wherein the electrode data from each of the two or more electrodes is sent, via the communication unit, to an Internet of Things (IoT) cloud server configured to interact with one or more IoT-capable devices.
77. The plant sensor of item 76, wherein the electrode data from each of the two or more electrodes are sent, via the communication unit, to an external device.
78. The plant sensor of item 74, wherein the instructions are configured to select one or more calibration plots to analyze at least one electrode data.
79. The plant sensor of item 78, wherein the instructions are configured to perform a signal calibration of at least one electrode.
80. The plant sensor of item 79, wherein the calibration comprises a pH-based signal calibration to determine a pH value from a voltage measurement.
81. The plant sensor of item 80, wherein the plant sensor is configured to detect a pH in a range of about 1 to 14, or pH in a range of about 2 to 13.
82. The plant sensor of item 69, wherein the plant sensor has a sensitivity of at least 1 mV/pH, at least 2 μmV/pH, or at least 3 μmV/pH.
83. The plant sensor of item 69, wherein the sensor is configured to detect a signal with a deviation of less than 10%, or less than 5%, or less than 1%, or less than 0.5%, or less than 0.1%, wherein the deviation is across at least three repeated measurements.
84. A method for continuously measuring pH in a plant, comprising:
attaching to the plant:
   a reference electrode (RE);
   a working electrodes (WE) configured to detect ions,
wherein each electrode comprises a plurality of microneedles, and
wherein each electrode is operatively connected to a voltage booster and an electrode control unit;
measuring an output voltage;
determining the pH based on a pre-determined calibration plot.
85. The plant sensor of item 84, wherein the RE is coated with Ag/AgCl paste.
86. The plant sensor of item 84, wherein the WE is coated with a PANI.
87. The method of item 84, wherein the electrode control unit comprises a processor, a voltage detector, and a voltage converter.
88. The plant sensor of items 84-87, wherein the sensor is configured to detect a signal with a deviation of less than 10%, or less than 5%, or less than 1%, or less than 0.5%, or less than 0.1%, wherein the deviation is across at least three repeated measurements.
89. The method of items 84, wherein the continuous measurement occurs for at least 120 days, or at least 90 days, or least 60 days, or at least 30 days, or at least two weeks, or at least 10 days, at least 7 days, or 1 to 7 days.
90. The method of items 84, wherein the electrode control unit further comprises a communication unit.
91. The method of item 90, wherein the electrode data from the electrodes is sent, via the communication unit, to an Internet of Things (IoT) cloud server configured to interact with one or more IoT-capable devices.
92. The method of item 91, wherein the electrode data from the electrodes are sent, via the communication unit, to an external device.
93. The method of item 92, wherein the calibration comprises a pH-based signal calibration to determine a pH value from a voltage measurement.
94. The method of items 84-93, wherein the electrodes are attached to a leaf of the plant or a stem of the plant.
95. The method of items 84-94, wherein the electrodes are attached to at least two locations of the same plant.
96. The plant sensor of any one of items 1-41 and 69-83, further comprising one or more screen printed electrodes. 97. The method of any one of items 42-67 and 84-95, further comprising one or more screen printed electrodes. 98. The flexible plant sensor of item 96, wherein the screen printed electrodes comprise a reference electrode (RE), a counter electrode (CE), and at least one working electrode (WE).
99. The flexible plant sensor of item 96, wherein the working electrode comprises an ethylene sensor.
100. The method of item 97, wherein the screen printed electrodes comprise a reference electrode (RE), a counter electrode (CE), and at least one working electrode (WE).
101. The method of item 97, wherein the working electrode comprises an ethylene sensor.
102. A biocompatible polymer plant sensor, comprising:
a substrate;
at least three sidewalls each attached to the substrate on a first side; and
a chamber;
wherein the chamber is enclosed by the at least three sidewalls and the substrate,
one or more sensors comprising a plurality of microneedles disposed on a second side of at least one sidewall, the second side being opposite to the first side attached to the substrate, and
one or more sensors disposed in the chamber,
wherein:
a) the microneedles have a height dimension of 100 μm to 5,000 μm and a base-width dimension of 100 μm to 5,000 μm;
b) the microneedles have a vertex angle of 3° to 90°;
c) the microneedles have a bending angle of less than 150 at a pressure of 600 kPa;
d) the microneedles have a height dimension of 100 μm to 5,000 μm and a base-width dimension of 100 μm to 5,000 μm and the microneedles have a vertex angle of 3° to 90°;
e) the microneedles have a height dimension of 100 μm to 5,000 μm and a base-width dimension of 100 μm to 5,000 μm and the microneedles have a bending angle of less than 15° at a pressure of 600 kPa;
f) the microneedles have a vertex angle of 3° to 90° and the microneedles have a bending angle of less than 150 at a pressure of 600 kPa; or
g) the microneedles have a height dimension of 100 μm to 5,000 μm and a base-width dimension of 100 μm to 5,000 μm, the microneedles have a vertex angle of 3° to 90°, and the microneedles have a bending angle of less than 150 at a pressure of 600 kPa.
103. The biocompatible polymer plant sensor of item 102, wherein the chamber is open to the surroundings on at least one side.
104. The biocompatible polymer plant sensor any one of items 102-103, further comprising a fourth sidewall.
105. The biocompatible polymer plant sensor any one of items 102-104, wherein three sidewalls have one or more sensors comprising a plurality of microneedles.
106. The biocompatible polymer plant sensor of any one of items 102-105, wherein the biocompatible polymer plant sensor is configured to interface with a drone.
107. The biocompatible polymer plant sensor of any one of items 102-106, wherein the one or more sensors disposed in the chamber are screen printed sensors.
108. The biocompatible polymer plant sensor of any one of items 102-107, wherein the screen printed electrodes comprise a reference electrode (RE), a counter electrode (CE), and at least one working electrode (WE).
109. The biocompatible polymer plant sensor of 108, wherein the working electrode is a dual working electrode.
110. The biocompatible polymer plant sensor of 108, wherein the working electrode comprises an ethylene sensor.
111. The plant sensor of any one of items 1-41 and 69-83, wherein the selectivity for target analytes is at least 1.1× higher than one or more interfering species.
112. The method of any one of items 42-67 and 84-95, wherein the selectivity for target analytes is at least 1.1× higher than one or more interfering species.
113. The plant sensor of any one of items 1-41 and 69-83, wherein a peak current value detected has a decrease of 2.5% or less over at least seven days.
114. The method of any one of items 42-67 and 84-95, wherein a peak current value detected has a decrease of 2.5% or less over at least seven days.
115. The biocompatible polymer plant sensor of item 102, wherein the selectivity for target analytes is at least 1.1× higher than one or more interfering species.
116. The biocompatible polymer plant sensor of item 102, wherein a peak current value detected has a decrease of 2.5% or less over at least seven days.

### EXAMPLES

The following examples are intended to exemplify the present disclosures and are not limitations of the claimed invention. All sensors, components, methods, assays, properties, and results disclosed in the examples form part of the present disclosure as if recited in the general disclosure and available for claiming as part of the invention.

### Example 1

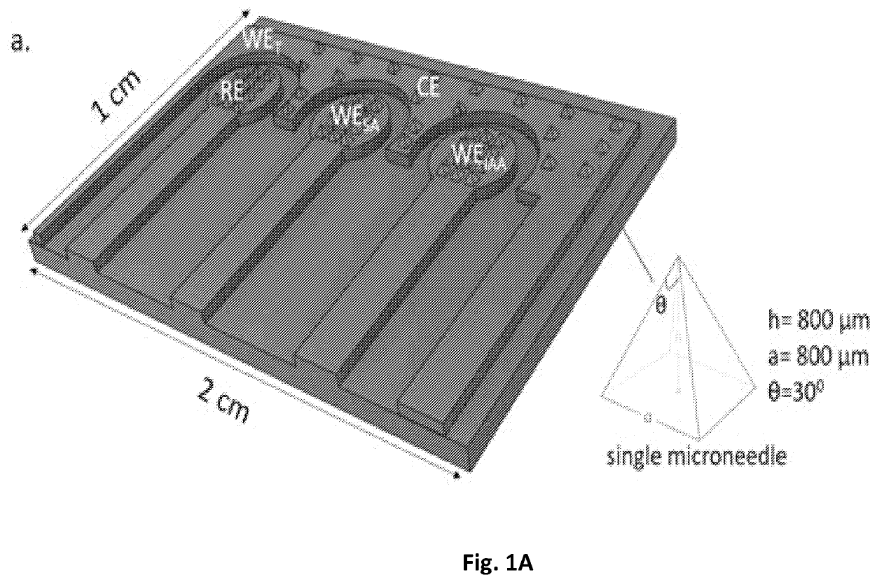

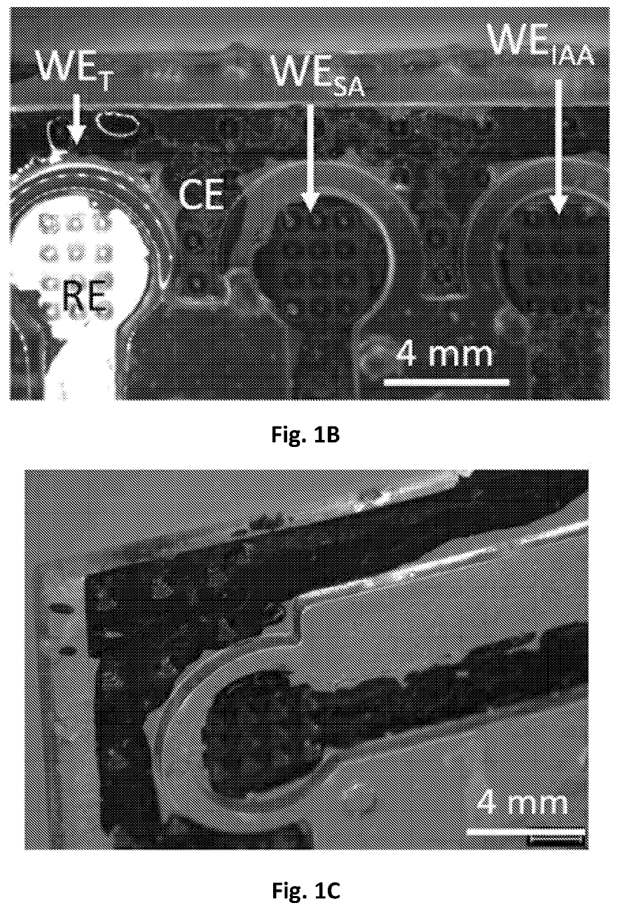

Formation of microneedle electrodes for a plant sensor. Each electrode was made of an array of square-based pyramid-shaped microneedles with a height of 800 μm, a base width of 800 μm, and each side making a 30° angle with the tip (FIG. 1A). The entire platform was designed with a stereolithography 3D printer using a biocompatible resin. The following working electrodes (WE): WE_{SA}, WE_{IAA}, W_{ET}, and the counter electrode (CE) were coated with graphene ink, while the reference electrode (RE) was covered with Ag/AgCl paste and subsequently cured at 100° C. for 60 minutes. FIGS. 1B and 1C illustrates the microscopic images of the fabricated multisensory platform carrying the microneedle electrodes. The plant sensor was comprised of three-electrode-based electrochemical sensors with a dimension of 2 cm×1 cm.

### Example 2

### Formation of Microneedle Electrode Coatings.

The WE_{SA} was coated with a copper-based metal-organic framework (CuMOF) for SA detection, WE_{IAA} was coated with gold nanoparticle decorated graphene hydrogel nanocomposite (AuNP-GH) for IAA detection, and W_{ET} with poly(3,4-ethylenedioxythiophene) polystyrene sulfonate (PEDOT:PSS) crosslinked by 3-glycidyloxypropyl)trimethoxysilane (GOPS) for temperature detection.

The CuMOF coating was synthesized by dissolving 0.4 g of polyvinyl pyrrolidone (PVP) in 8 μmL of dimethylformamide (DMF) and 8 μmL of ethanol. Next, a homogeneous mixture of 46.6 μmg of copper nitrate hydride and 10.8 μmL of 2-amino terephthalic acid was prepared in 4 μmL of DMF, which was added to the previously prepared PVP solution and heated at 100° C. for 5 hours. The resulting green residue was dissolved in 40 μmL of DMF and heated in an oven at 100° C. for 8 hours. The solution was cooled down to room temperature and subsequently centrifuged at 1000 rpm for 30 μminutes. The resulting CuMOF precipitates were collected and dried at 100° C. The CuMOF powders were added to carbon black (CB) at a weight ratio of 2:1 and dissolved in deionized water at a 2 μmg/mL concentration. After adding 0.01% w/v of Nafion, the solution was sonicated for 30 minutes. Subsequently, 1 μL of the resulting nanocomposite solution was drop coated on the WE_{SA} electrode surface.

The AuNP-GH was synthesized according to the method described in (X. Cao, et al., “Gold nanoparticle-doped three-dimensional reduced graphene hydrogel modified electrodes for amperometric determination of indole-3-acetic acid and salicylic acid”, Nanoscale, 11, 10247-10256 (2019), which is incorporated herein by reference in its entirety). Briefly, 1 μmg mL−1 of graphene oxide suspension was prepared. 20 μmL of this suspension was added to 2 μmL of 0.8511 mg mL⁻¹ chloroauric acid solution and 1 μmL of triethylenetetramine. The resulting mixture was sonicated for 10 μminutes and then heated at 140° C. for 12 h. The obtained AuNP-GHs were cooled down to room temperature and then freeze-dried for 24 h to form powders that were stored in a desiccator.

To print the temperature sensing layer, 100 μmg of PEDOT:PSS solution and 50 μmg of non-ionic surfactant Triton X-100 solution (1.3 wt % in DI water) were mixed with GOPS (at a GOPS to PEDOT:PSS weight ratio of 9:1). The resulting mixture was centrifuged for 15 μmin and degassed for 5 μmin. The solution was drop cast on the working electrode, W_{ET} (FIG. 1A) followed by annealing at 140° C. in air for 30 μmin to remove the solvent and enable the cross-linking process. Finally, a 25 μm-thick Kapton tape was used as the encapsulation layer to cover the PEDOT:PSS coating.

### Example 3

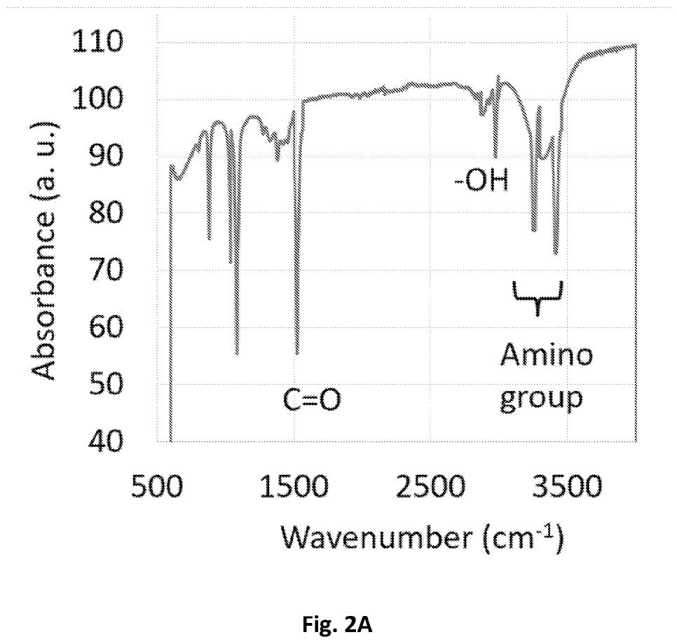

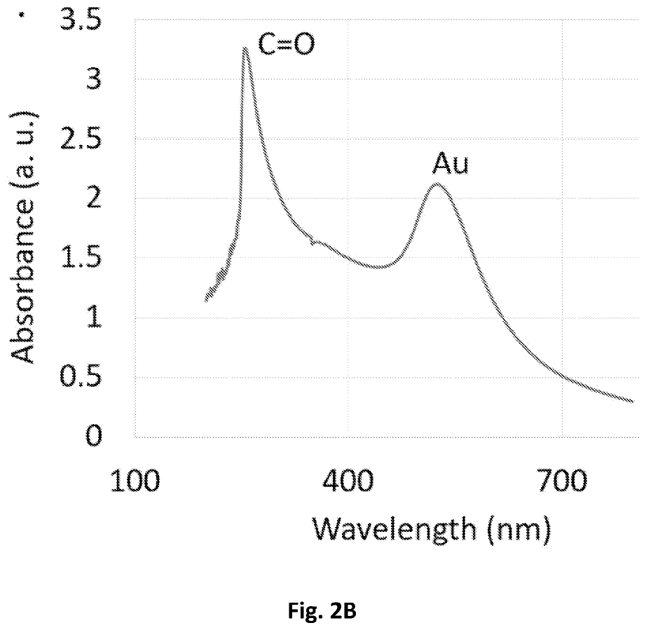

Characterization of microneedle electrode coatings. The CuMOF coating was characterized by Fourier Transform Infrared Spectroscopy (FTIR), as is demonstrated in FIG. 2A. The FTIR spectrum confirmed the presence of amino groups and —OH and C═O functional groups. The AuNP-GH coating was characterized by ultraviolet-visible (UV-Vis) spectroscopy, which indicated the presence of Au molecules and C═O bonds (FIG. 2B).

EmStat 3 potentiostat (BASi, Inc.) was used as the electrode control unit to perform the electrochemical measurements of SA and IAA according to standard manufacturer's protocols. The onboard temperature sensor was resistive, generating resistance variations in response to varying leaf temperatures. A separate ESP32 feather development board was used as a data acquisition system (DAS) to acquire and process the temperature sensor measurements. The temperature sensor was connected in series with a known resistor, and a built-in analog to digital converter on the ESP32 board measured the analog voltages across the sensor. The measured resistance values across the temperature sensor were further verified with a benchtop LCR meter (Applent, AT 3817) according to standard manufacturer's protocols.

### Example 4

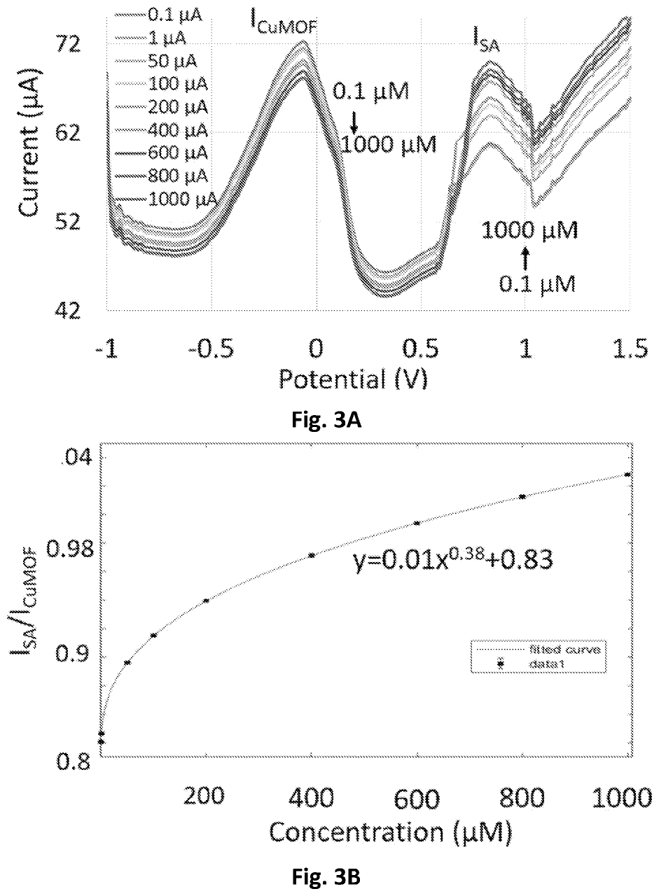

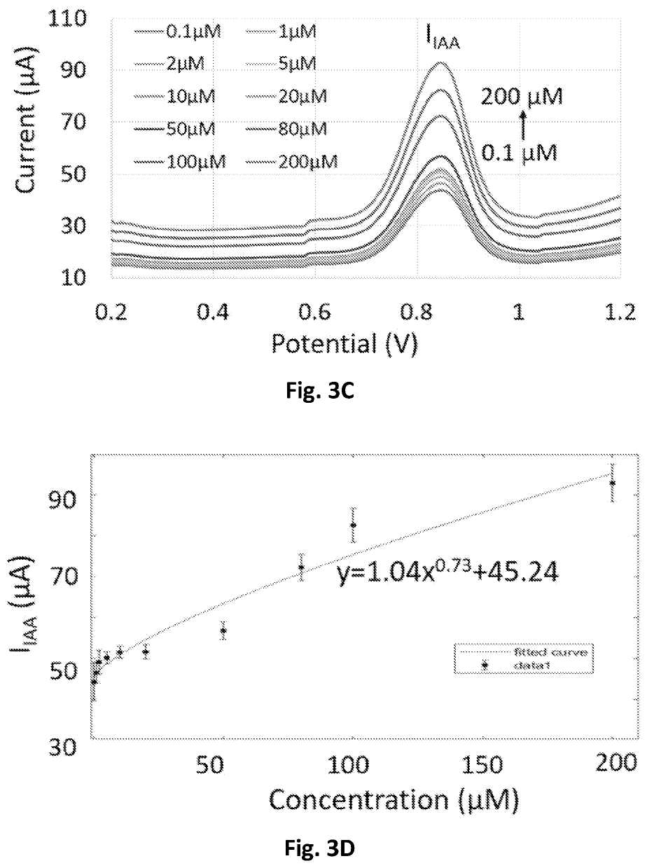

Electrochemical detection of SA and IAA hormones. Differential pulse voltammetry (DPV) was employed for electrochemical measurements of varying SA and IAA levels. The sap was collected from live cabbage plants and spiked with hormones to prepare the SA and IAA concentrations depicted in FIGS. 3A and 3C. In the potential range from −1V to 1.5V, the reduction peak current for CuMOF occurred at around −0.06 V, while the oxidation peak current of SA appeared at 0.82 V (FIG. 3A). Owing to the reasonable separation between the redox potentials, the ratio of the two peak currents was plotted against the SA levels to generate the calibration plot in FIG. 3B. Next, the IAA redox peak current at ˜0.85 V (FIG. 3C) was plotted against the IAA concentrations to generate the calibration plot in FIG. 3D. With increasing SA/IAA levels in sap, the redox peak currents for SA/IAA increased due to the oxidation of the hormones by the selective coating. The sensitivity of the SA sensor was calculated as 0.005 μM−1 with a detection limit of 1 μM. The sensitivity of the IAA sensor was calculated as 0.76149 μA μM−1 with a detection limit of 0.1 μM.

### Example 5

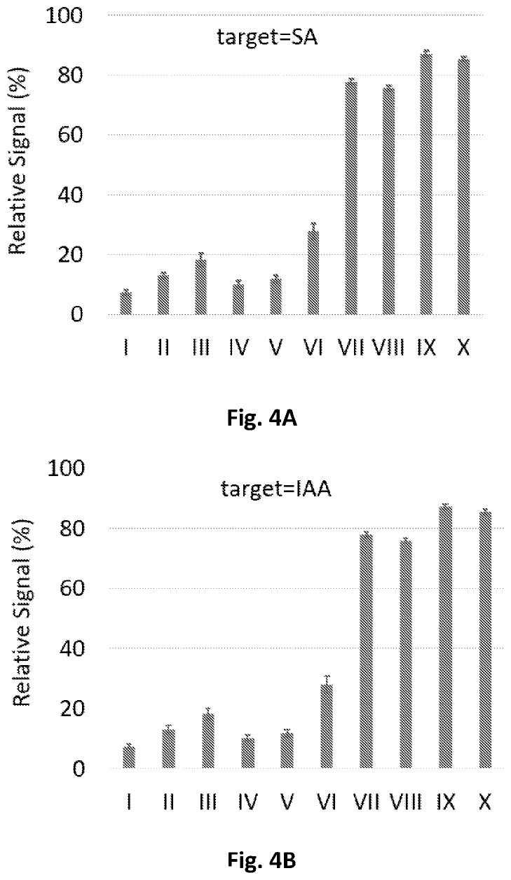

Selectivity, Reproducibility, and Repeatability Studies. The hormone sensors exhibited good selectivity against common interfering species found in sap, as depicted in FIGS. 4A-4B. The sensors were tested against different interfering species (e.g., Jasmonic acid, L-Cysteine, glucose, citric acid, and ascorbic acid) and their mixture, commonly found in the plant sap. It was observed that the sensors demonstrated a much higher relative signal than the interfering species present in the sap. The relative signal was defined by the following Equation 1:

$$Relative Signal = ( {R}_{a} - {R}_{b} ) / {R}_{b} \qquad ( 1 )$$

Rₐ represents the ratio of the redox peak currents of the analyte and the CuMOF coating. R_{b} represents the ratio of the base current to the CuMOF peak current of a blank solution. In FIGS. 4A-4B I-X denote: (I) Jasmonic acid (JA)=50 μM, (II) L-Cysteine (L-Cys)=50 μM, (III) glucose=50 μM, (IV) citric acid=50 μM, (V) ascorbic acid=50 μM, (VI) a mixture of JA, L-Cys, glucose, citric acid, and ascorbic acid (50 μM each), (VII) target hormone=100 μM, (VIII) a mixture of ascorbic acid, JA, L-Cys, glucose, citric acid, ascorbic acid (50 μM each), and target=100 μM, (IX) target (SA=900 μM or IAA=200 μM), (X) a mixture of ascorbic acid, JA, L-Cys, glucose, citric acid, ascorbic acid (50 μM each), and target (SA=900 μM or IAA=200 μM).

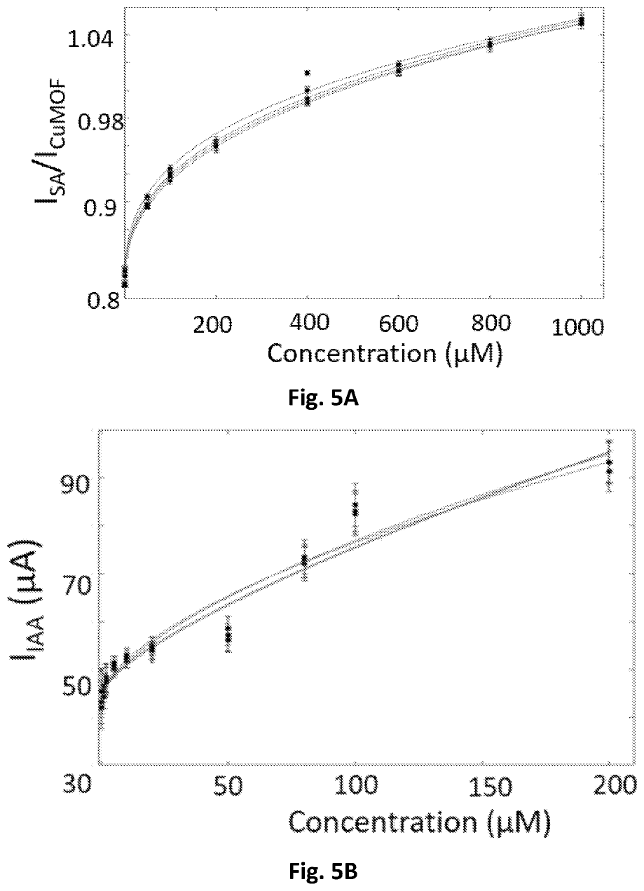

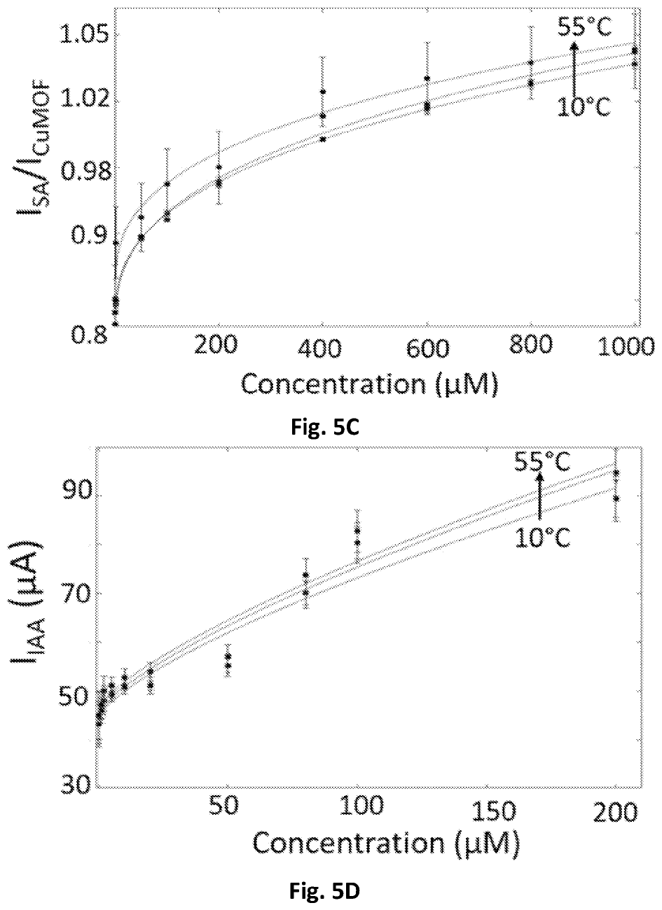

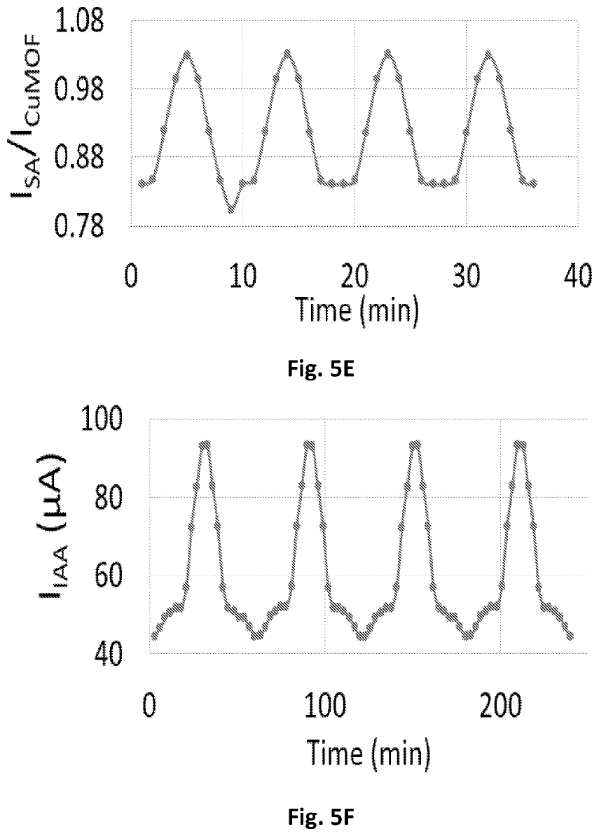

The hormone sensors also demonstrated excellent reproducibility for 4 repeated measurements with less than 1% deviation (FIGS. 5A-5B). In addition, with increased temperature, the sensor calibration curves shifted upwards with less than 10% deviation (FIGS. 5C-5D). Hence, temperature correction was done with an in-built temperature sensor. The sensors also demonstrated repeatable characteristics under cyclic variations in hormone levels (the same concentrations used for calibration), indicating the feasibility of field deployment (FIGS. 5E-5F).

### Example 6

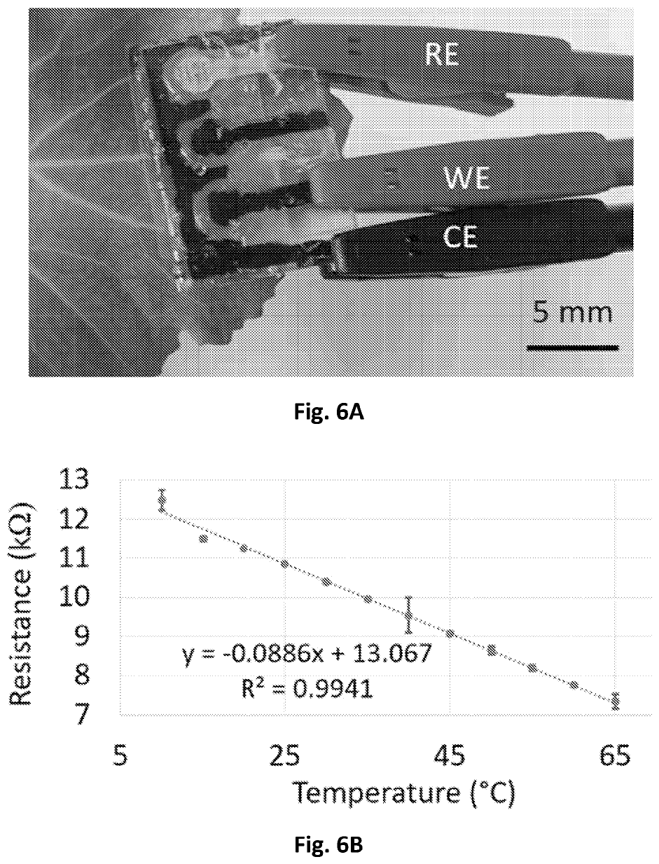

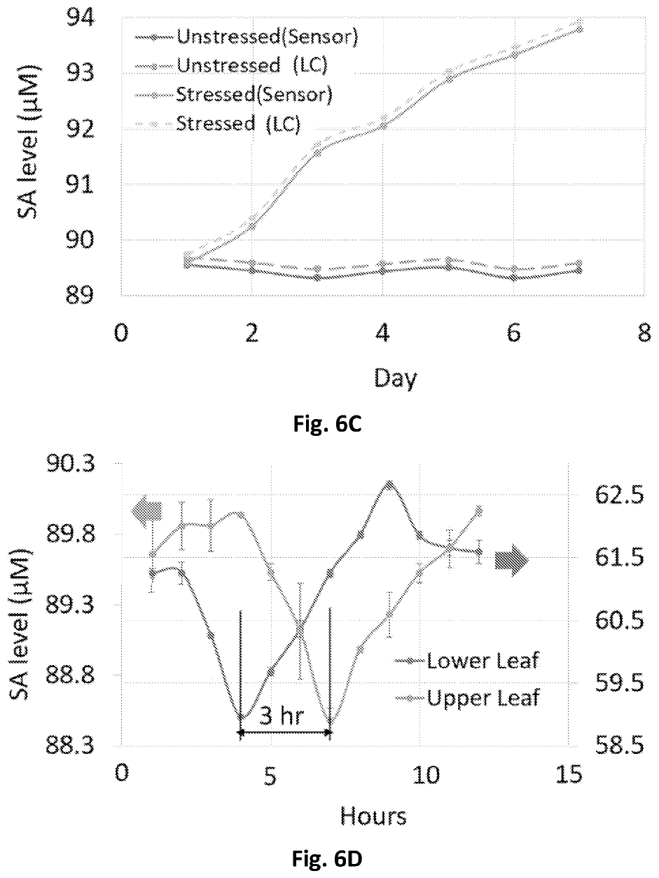

Real-time hormone measurements with temperature correction. The microneedle sensor was mounted on the leaf of an unstressed (control) and a water-stressed cabbage plant (FIG. 6A), and SA levels were measured. The stressed plant was not irrigated for three days. The unstressed plant was irrigated 10 hours before the test. FIG. 6B shows the calibration plot of the onboard temperature sensor. FIG. 6C shows the temperature-corrected real-time SA levels of water-stressed and control plants measured over 7 days with the microneedle sensor. A progressive increase in the SA level was observed in the stressed plant. Next, two sensors were mounted at different heights (0.5 and 6.5 cm) of the same plant. The sensors accurately measured the SA dynamics across the plant, as shown by the difference in the SA rise time (3 hours) captured by the leaf sensors (FIG. 6D). The measurements were repeated with three plants and confirmed by the liquid chromatography (LC) tests (Control Tests). As shown in FIG. 6C, the hormone levels measured with the gold standard LC Control Tests were very close to the levels measured with the plant sensor, thereby validating the accuracy of the microneedle sensor.

### Example 7

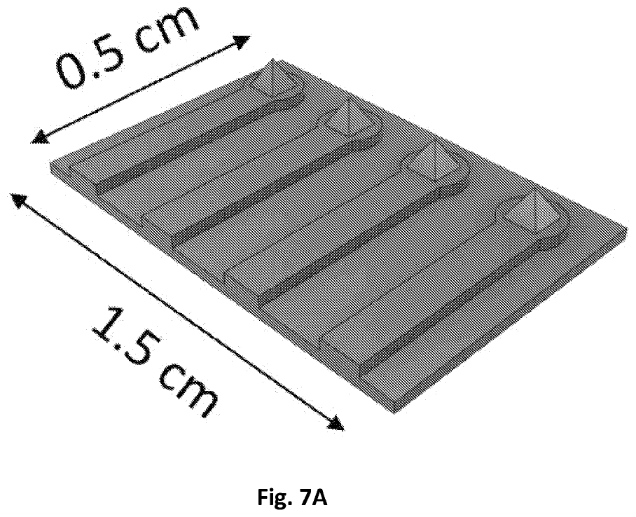

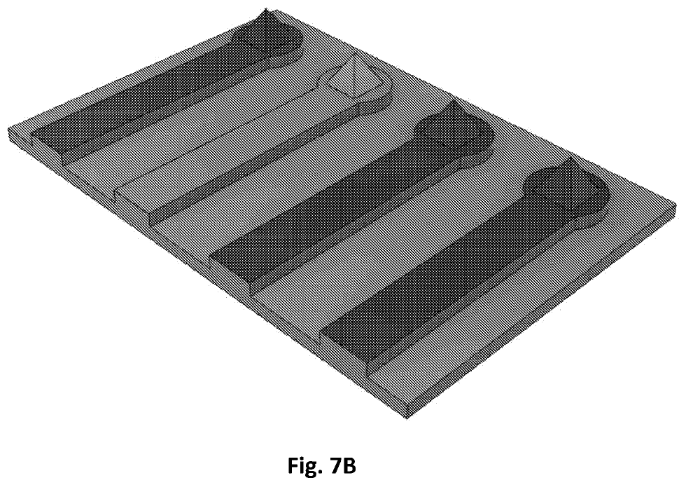

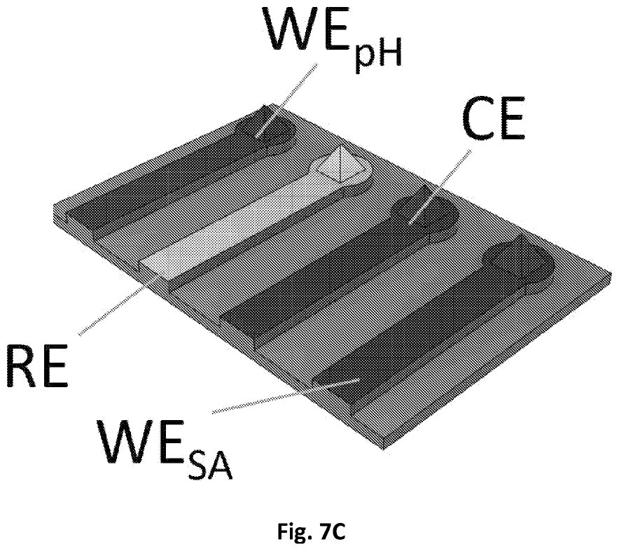

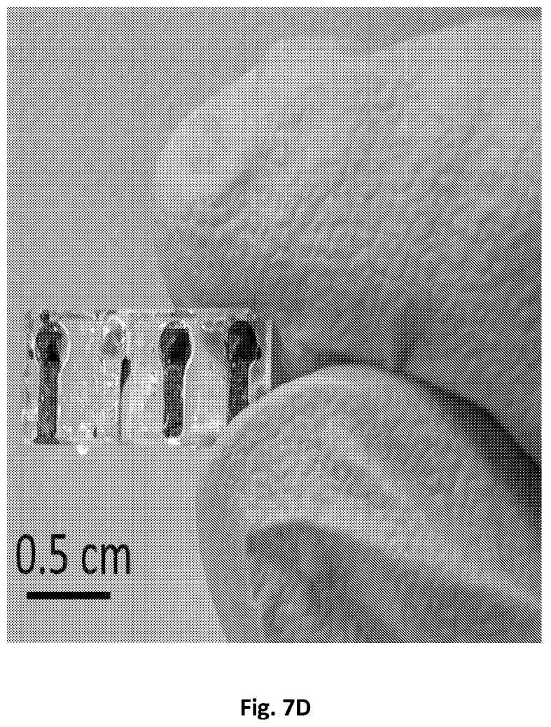

Formation of microneedle electrodes for a plant sensor. A separate plant sensor was prepared as describe below. The device was prepared as a four-electrode electrochemical sensor with two working electrodes (WE_{SA} and WE_{pH}) for measuring SA and pH levels in a plant stem, one shared counter electrode (CE), and one shared reference electrode (RE). Each two-dimensional (2D) electrode surface contained a protruded pyramid-shaped microneedle, as shown in FIG. 7A. The entire device was printed with the Form 3B (FormLabs. Inc.) stereolithography 3D printer according to standard manufacturer's protocols. A biocompatible resin (BioMed) was used as the material for the microneedle structure to reduce the chance of biofouling. The device was designed using AutoCAD Fusion 360 software. The design was then exported to the 3D printer. The printed microneedles were pyramidal shaped with a square base of 800 μm and a height of 2000 μm. After printing, the structure was washed with isopropyl alcohol with constant stirring for 30 μminutes. Next, the device was cured under ultraviolet light at 60° C. for 60 μminutes, resulting in the 3D device depicted in FIG. 7A. The working electrodes: WE_{SA} and WE_{pH}, and the counter electrode (CE) were coated with graphene ink (FIG. 7B), while the reference electrode (RE) was covered with Ag/AgCl paste and subsequently cured at 100° C. for 60 μminutes (FIG. 7C). The entire device had a length of 1.5 cm and a width of 0.5 cm (FIG. 7D).

### Example 8

Synthesis of Selective Coating for the SA Sensor and coating for the pH sensor. The SA sensor was prepared by coating the working electrode, WE_{SA}, with a copper-based metal-organic framework (CuMOF) according to Example 2, above.

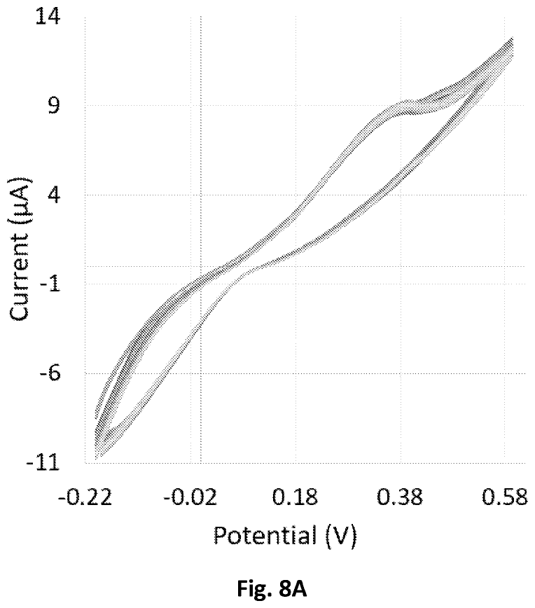

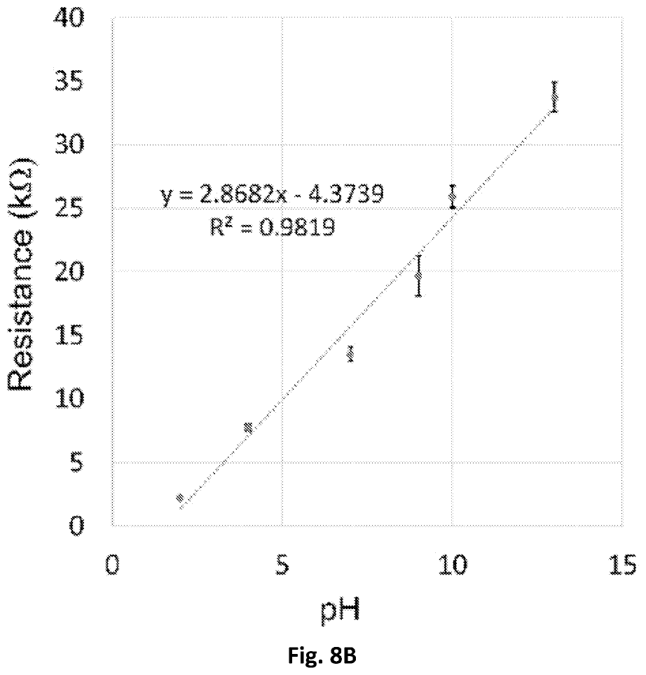

The pH sensor comprised one working electrode, WE_{pH}, and the reference electrode RE (FIG. 7C). The WE_{pH} was coated with a polyaniline (PANI) based nanofiber, sensitive to hydronium (H₃O⁺) ions. In addition, the PANI coating enhanced the electrode surface area and introduced reproducible and biocompatible characteristics to the sensor. The PANI coating was formed by sonicating 1M of aniline in 1M of HCl for 1 hr to make a homogeneous solution. The WE_{pH} and RE were immersed in the resulting solution, and cyclic voltammetry (CV) was run in the potential range from −0.2 V to 0.6 V and at a scan rate of 40 μmV/s for 50 cycles (FIG. 8A).

pH sensor calibration and data acquisition. The pH sensor was calibrated with plant sap at varying pH levels, as is shown in FIG. 8B. The sap was extracted from the stem of cabbage plants using standard extraction tools. Consequently, the extracted sap was centrifuged at 1000 rpm for 1 hour to precipitate the debris and other solid compounds. The supernatant (which contained proteins and metabolites) was collected and stored at −1° C. for future use. The original sap had a pH of 4.09. Different volumes of 0.01 μM sodium hydroxide and 0.01M HCl were mixed with the original sap solution (pH=4.09) to prepare solutions of varying pH levels, i.e., pH=2.1, 7.1, 9.4, 10.14, and 13.04).

An EmStat 3 potentiostat was used as described above in Example 3. The developed pH sensor was resistive, generating resistance variations in response to different sap pH levels. A data acquisition system (DAS) was designed to acquire and process the pH sensor measurements. An ESP32 feather development board was used for this purpose. The pH sensor was connected in series with a known resistor, and a built-in analog to digital converter on the ESP32 board measured the analog voltages across the pH sensor. The measured resistance values across the pH sensor were further verified with a benchtop LCR meter (Applent, AT 3817) according to manufacturer's protocols.

### Example 9

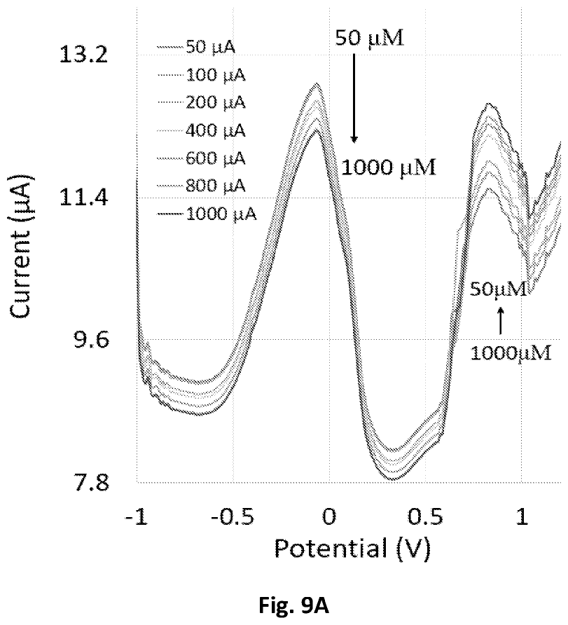

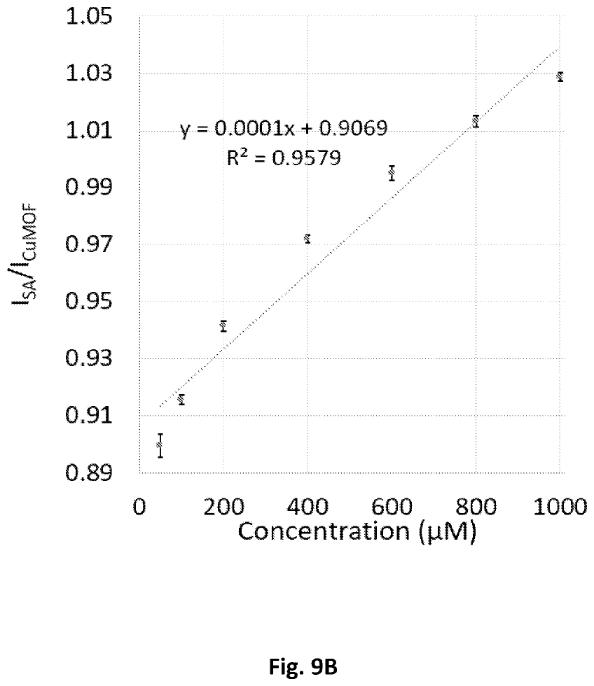

Electrochemical Detection of SA. Differential pulse voltammetry (DPV) technique was used to conduct the electrochemical measurements in the potential range from −1V to 1.2V and at a scan rate of 50 μmV/s with the potential step, pulse amplitude, and pulse duration being 0.01V, 0.3V, and 0.1 s, respectively. Sap was collected from live cabbage plants and spiked with SA to prepare seven concentrations: 50 μM, 100 μM, 200 μM, 400 μM, 600 μM, 800 μM, and 1000 μM. The DPV plots for varying SA levels in FIG. 9A show that the CuMOF redox peak was located at −0.06 V and the SA peak at 0.83 V. With increasing SA concentrations in sap, the CuMOF peak current decreased while the SA peak current increased owing to the oxidation of SA by the CuMOF coating. Due to a reasonable amount (i.e., 0.89V) of separation between the CuMOF and SA peaks, the ratio of the two peak currents was considered a response signal. The resulting calibration curve (I_{SA}/I_{CuMOF} vs. concentration) is shown in FIG. 9B for sap pH=7.1. The sensor showed a linear response with R²=0.9579. The sensor's sensitivity was calculated as 0.0001 μM⁻⁴ with a detection limit of 37.4 μM.

### Example 10

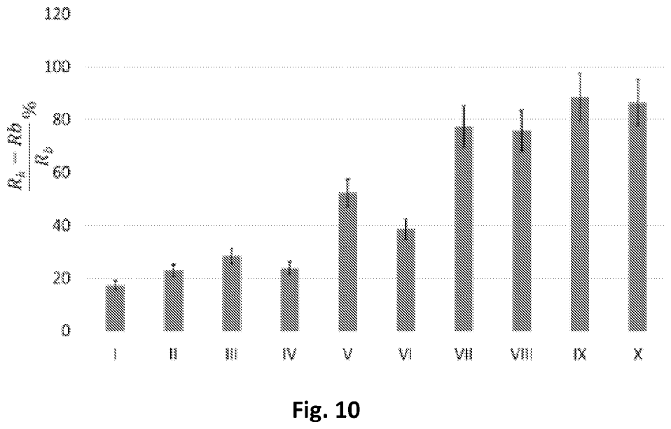

Selectivity Test. The sensor was tested against different interfering species commonly found in the plant sap. The following solutions were used for the selectivity test: (i) 40 μM JA, (ii) M L-Cysteine (L-Cys), (iii) 40 μM glucose, (iv) 40 μM citric acid, (v) 40 μM ascorbic acid, (vi) a mixture of 40 μM JA, 40 μM L-Cys, 40 μM glucose, 40 μM citric acid, and 40 μM ascorbic acid, (vii) 100 μM SA, (viii) a mixture of 40 μM JA, 40 μM L-Cys, 40 μM glucose, 40 μM citric acid, 40 μM ascorbic acid, and 100 μM SA, (ix) 900 μM SA, and (x) a mixture of 40 μM JA, 40 μM L-Cys, M glucose, 40 μM citric acid, 40 μM ascorbic acid, and 900 μM SA. As shown in FIG. 10, the sensor demonstrated a much higher relative signal than the interfering species present in the sap. The relative signal was defined by the following Equation 1.

### Example 11

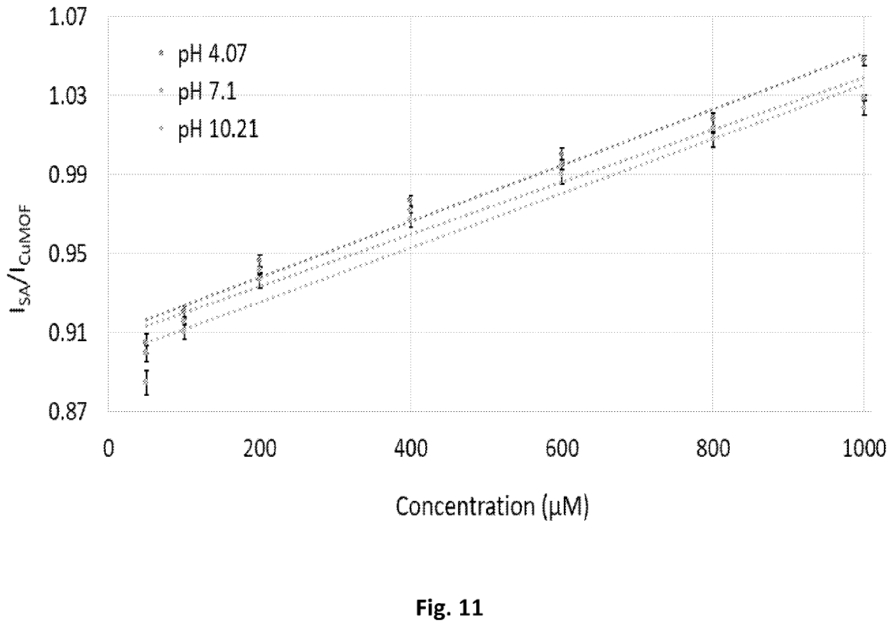

pH correction of the SA sensor. The pH of sap varies with the growth of the plant and environmental stress conditions. Hence, it is crucial to correct the measured SA levels under varying pH values. The SA sensor was calibrated with sap solutions having different pH values: 4.09, 7.1, and 10.14. FIG. 11 shows that the calibration plot shifted toward lower I_{SA}/ICuM_{OF} with increasing pH. This can be attributed to the presence of negative charges on the working electrode surface originating from the CuMOF coating. With increasing pH, the solution was less positive, resulting in a decrease in the effective electrostatic interactions between the charges and the CuMOF-modified electrode. The sensor demonstrated good performance under varying pH conditions. The calibration plots with a slope of 0.0001±0.0002 μM¹ and intercept of 0.9069±0.0125 were measured within a linear pH range (from pH=4.09 to 10.14). Considering pH=7.1 as the reference, the correction factors for slope and intercept (i.e., fₛₗₒₚₑ and f_{intercept}) under different pH environments were calculated using Equations 2 and 3, below:

$${f}_{slope} = \frac{slope ( pH )}{slope ( 7.1 )} \qquad ( 2 )$$

$${f}_{intercept} = \frac{intercept ( pH )}{intercept ( 7.1 )} . \qquad ( 3 )$$

### Example 12

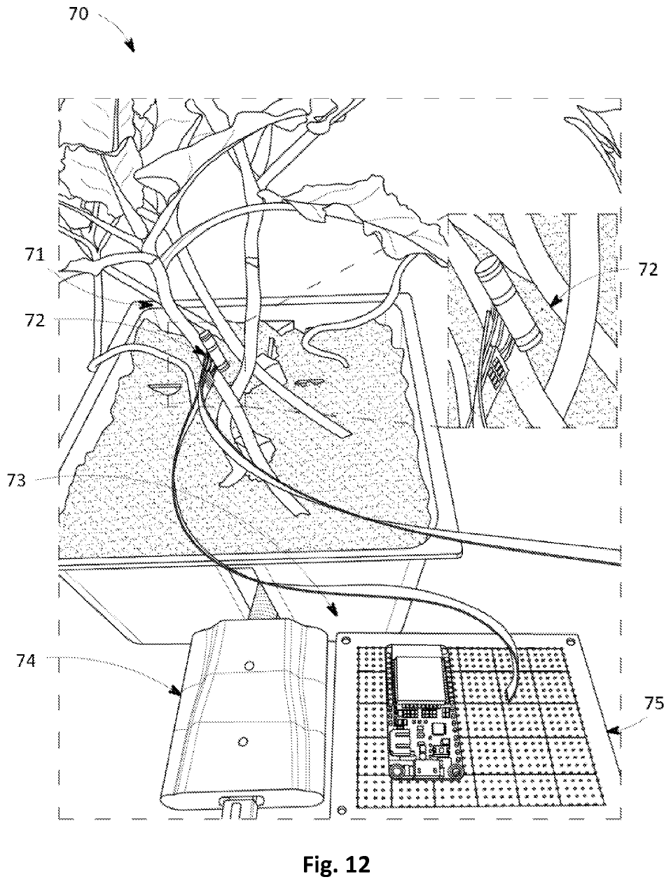

Real-time measurements in live plants. Three unstressed (control) and three water-stressed cabbage plants were used for real-time SA measurements. The stressed plants were not irrigated for three days. The unstressed plants were irrigated 10 hours before the test. The plant sensor was mounted on the stem of six cabbage plants as shown by an exemplary setup illustrated in FIG. 12, and SA levels were monitored every hour for an extended period of 12 hours. Specifically, the plant sensor system 70 included a sensor 72 attached to a plant stem 71 connected to an electrode control unit 73. The electrode control unit 73 included a potentiostat 74 and a data acquisition system 75 for determining pH corrected SA levels.

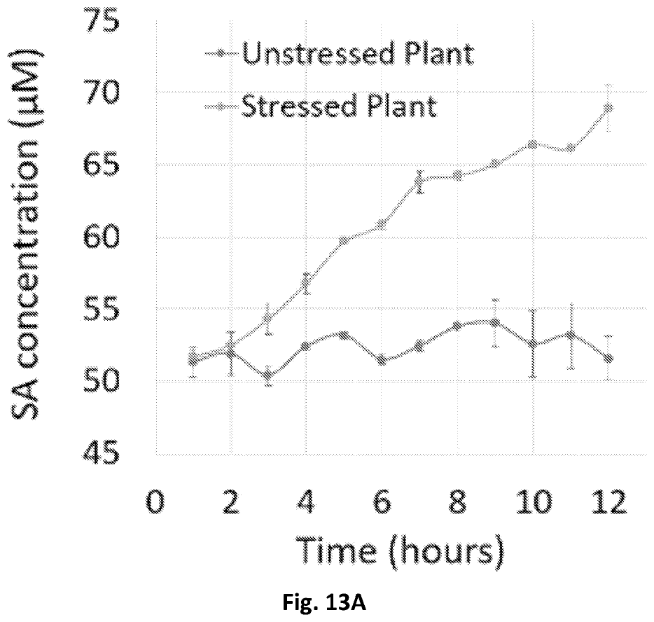

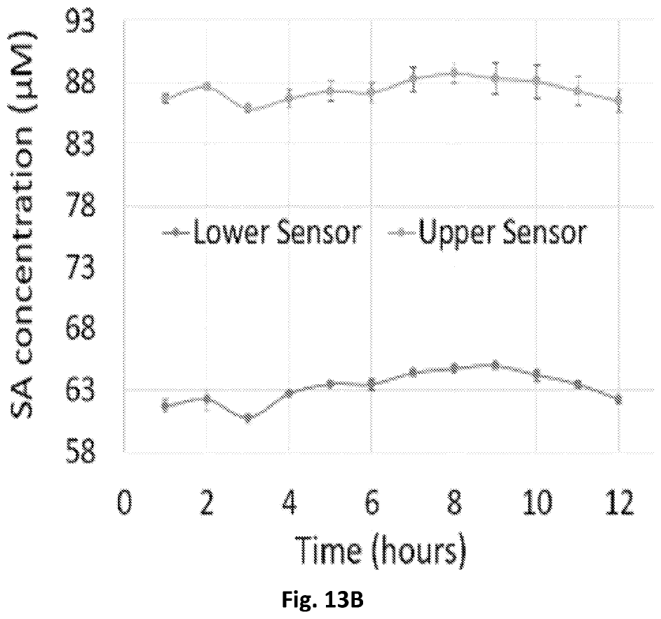

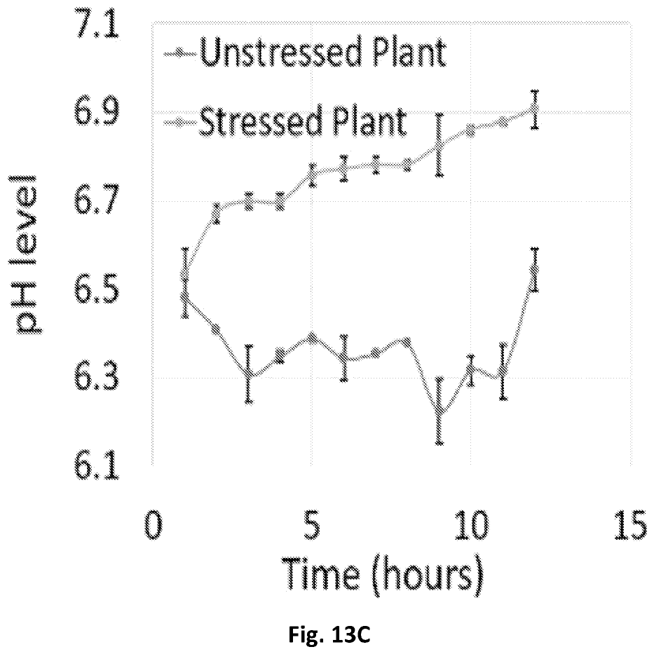

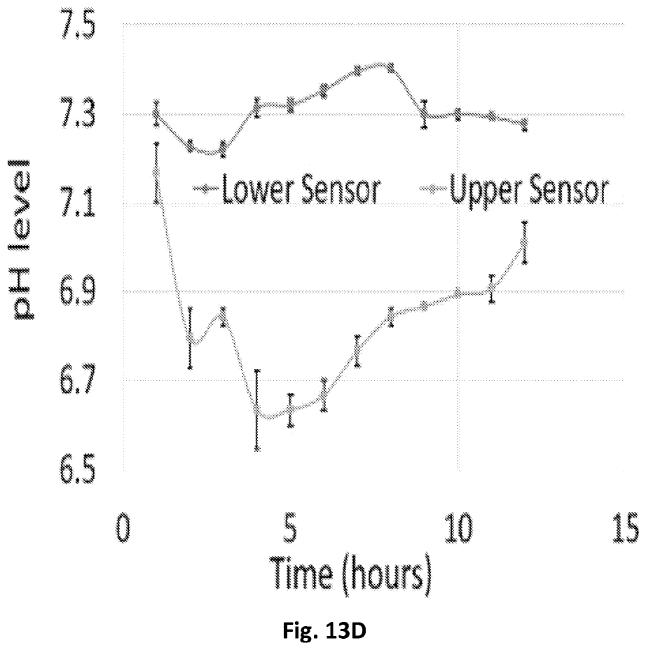

The pH corrected SA levels are shown in FIG. 13A for unstressed and water-stressed plants. FIG. 13A shows a rising trend in the SA levels in the water-stressed plant using the real-time in situ monitoring of phytohormones using the plant sensor. Raw data plotted in FIG. 13A for the SA measurements compared with ground truth measurements from Fourier Transform Infrared Spectroscopy (FTIR) is shown in Table 1.

TABLE 1

| Unstressed | | | Stressed | | |
|---|---|---|---|---|---|
| Sensor | FTIR | Percent Dev. | Sensor | FTIR | Percent Dev. |
| 51.33057 | 52.81 | −2.80143 | 81.6873 | 81.17 | 0.637304 |
| 51.90433 | 52.51 | −1.15343 | 82.51053 | 81.68 | 1.016814 |
| 50.408 | 52.81 | −4.54839 | 84.32333 | 83.26 | 1.277124 |
| 52.39997 | 54.71 | −4.22232 | 86.78267 | 85.51 | 1.488325 |
| 53.183 | 55.8 | −4.68996 | 89.66347 | 88.21 | 1.647735 |
| 51.51533 | 54.51 | −5.49379 | 90.88333 | 89.17 | 1.921423 |
| 52.43267 | 54.61 | −3.98706 | 93.819 | 91.32 | 2.736531 |
| 53.78233 | 55.45 | −3.00751 | 94.23033 | 91.78 | 2.66979 |
| 54.003 | 56.16 | −3.84081 | 95.039 | 92.78 | 2.434792 |
| 52.59973 | 55.16 | −4.64153 | 96.4136 | 93.98 | 2.589487 |
| 53.16467 | 56.51 | −5.9199 | 96.1518 | 93.85 | 2.452637 |
| 51.572 | 52.21 | −1.22199 | 98.92137 | 96.16 | 2.871638 |

The plant sensor was also mounted on the same plant at two different locations (one sensor placed near the root and the other placed near the apex). The plant was irrigated 10 hours before the test. FIG. 13B shows the SA measurements taken at two locations of the same plant over 12 hours. Raw data plotted in FIG. 13B for the SA measurements compared with ground truth measurements from Fourier Transform Infrared Spectroscopy (FTIR) are shown in Table 2. The SA levels measured with the plant sensors (FIGS. 13A-13B) were verified with ground truth measurements from Fourier Transform Infrared Spectroscopy (FTIR). A relative deviation of less than 500 was observed between the two measurements (Tables 1-2). The SA levels near the apex of the plant (7 cm above the soil surface) were higher than the SA levels near the root (1 cm above the soil surface). The corresponding pH level variations are shown in FIGS. 13C-13D. The difference in the SA levels was a function of the location of the sensor illustrating the hormone flow across the plant.

TABLE 2

| Lower | | | Upper | | |
|---|---|---|---|---|---|
| Sensor | FTIR | Percent Dev. | Sensor | FTIR | Percent Dev. |
| 61.65557 | 58.62 | 5.178381 | 86.6959 | 82.26 | 5.392536 |
| 62.22933 | 59.24 | 5.04614 | 87.63767 | 83.26 | 5.257827 |
| 60.733 | 57.85 | 4.983572 | 85.894 | 81.45 | 5.456104 |
| 62.72497 | 59.7 | 5.066946 | 86.71867 | 82.42 | 5.215563 |
| 63.508 | 60.4 | 5.145695 | 87.30413 | 83.01 | 5.173031 |
| 63.507 | 60.41 | 5.126635 | 87.197 | 82.82 | 5.284955 |
| 64.42433 | 61.23 | 5.216942 | 88.27833 | 83.81 | 5.331504 |
| 64.774 | 61.6 | 5.152597 | 88.732 | 84.32 | 5.232448 |
| 64.99467 | 61.82 | 5.135339 | 88.341 | 84.01 | 5.155339 |
| 64.25807 | 61.17 | 5.048335 | 88.08323 | 83.72 | 5.211698 |
| 63.48967 | 60.43 | 5.063158 | 87.296 | 82.95 | 5.239301 |
| 62.23033 | 59.28 | 4.976946 | 86.49453 | 82.25 | 5.160527 |

### Example 13

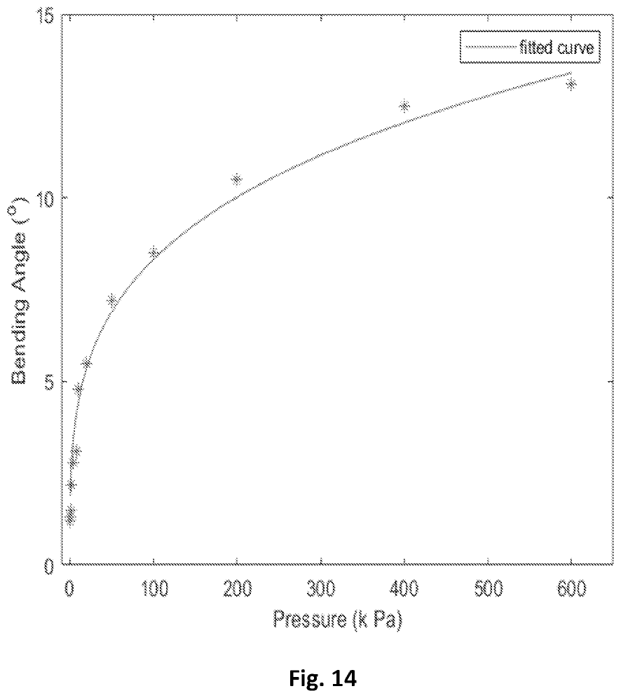

Stress-strain Property of Microneedles. The mechanical deformation of the microneedles on the plant sensor was characterized for varying applied pressure. The Platen press machine from Carver, Inc. was used to apply, measure, and control pressure. The bending angles of the microneedles were determined by capturing images of the bent needles with the Leica M205A microscope. The microneedles were subjected to a gradually increasing load (pressure) until failure occurred. FIG. 14 shows the resultant stress-strain behavior of the microneedles. As the pressure was increased, the bending angle of the microneedles increased. A power curve (i.e., y=ax^{b}+c) was fitted through the data points. At an applied pressure of 700 kPa, the needles completely broke (i.e., the failure point). As shown in FIG. 14, a bending angle of less than 15° was achieved at a pressure up to 600 kPa and a bending angle of 100 or less was achieved at a pressure up to 200 kPa. For all the hormone measurements, 5 kPa of pressure was found to be optimum (with the bending angle being 2.960) for the microneedles to penetrate the stem and access the xylem/phloem sap.

Any of the above protocols or similar variants thereof can be described in various documentation associated with a plant sensor product. This documentation can include, without limitation, instructions, protocols, statistical analysis plans, and other documentation that may be associated with a plant sensor product. It is specifically contemplated that such documentation may be physically packaged with a plant sensor product according to the present disclosure as a kit, as may be beneficial or as set forth by regulatory authorities.

### Example 14

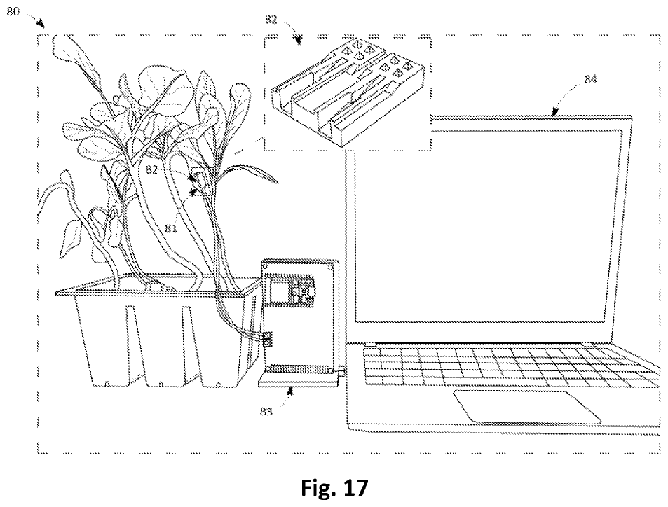

Microneedle patch plant sensor. A microneedle patch was designed using AutoCAD Fusion360 software and printed with FormLabs 3D printer using a bio-compatible resin. The patch was composed of two 2×2 arrays of microneedles. Each array was made on a 1 cm×1 cm square base (an example of which is shown in the inset of FIG. 17). The printed microneedles were pyramidal shaped with a square base of 900 μm, a height of 800 μm, and an angle of 30^{°} at the tip. The patch had two trenches extending from the square base of the microneedle array to the edge of the patch for making conductive traces. Both the trenches ramped up at an angle to the base of the needles to provide a solid support for the conductive wires and prevent open circuits when the patch was bent, twisted, dropped, or otherwise mishandled. One microneedle array was configured as a working electrode by coating the array with graphene. The other microneedle array was configured as a reference electrode by coating the microneedle array with Ag/AgCl. Subsequently the patch was cured at 100° C. for 60 μminutes. The working electrode was then coated with a polyaniline (PANI) based nanofiber, sensitive to hydronium (H₃O⁺) ions according to previously described methods. The microneedle patch sensor was thus configured to detect changes in voltage values measured between the two electrodes that were translated into variations in pH level.

FIG. 17 shows an exemplary IoT-enabled plant sensor 80. A sensor 82 (microneedle patch shown in the inset) is attached to the plant leaf 81 and the electrode control unit 83 (data acquisition and processing unit) receives the voltage reading, amplifies it by the voltage booster (not shown), processes the measured signal by a microcontroller (not shown), and transmits the data to the cloud system via a communication unit (not shown), allowing the data to be displayed on a data display device 84 (e.g., an external device).

### Real-Time Monitoring of Sap pH Levels.

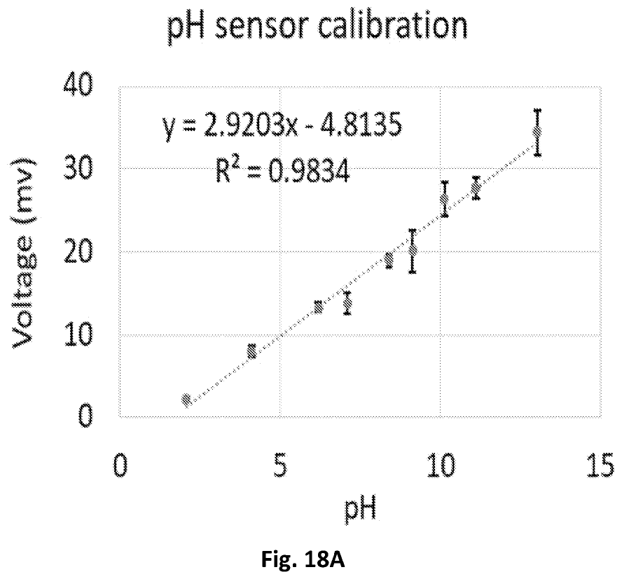

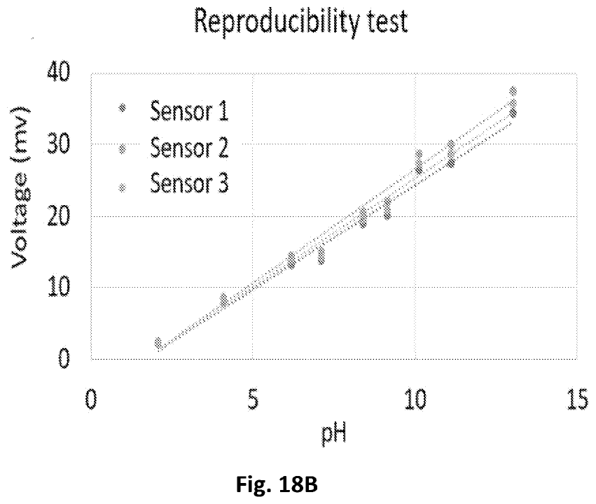

The microneedle-based pH sensor described above was calibrated for sap pH levels ranging from 2 to 13. The output voltage from the microneedle patch was stepped up 10 times by a voltage boost converter circuit comprising a 72 nH Inductor, a 1N4007G Diode, a 1.7 μF Capacitor, a 22 KΩ resistor, and an IRF540N MOSFET. The analog voltage was then converted to a digital signal by the in-built analog to digital converter in the ESP32 Wroom microprocessor unit. The microcontroller compared the measured voltage with a previously stored calibration curve to compute the corresponding pH value, which was sent to the InfluxDB database via Wi-Fi. A 555 timer was used to set the data acquisition frequency at 1 kHz. The whole system was powered by a 9V battery. The calibration curve was generated by plotting the voltage measured across the microneedle array as a function of pH levels (FIG. 18A). The calibration curve is linear with a sensitivity of 2.92 μmV/pH.

The open source analytics and interactive visualization web application, Grafana, was configured to display the pH readings transmitted by the leaf patch to the cloud system. The sap pH readings were displayed both in graphical and tabular formats on the data display device, allowing the user to view different data formats on the same dashboard. The system was also configured to set an alert. For example, if the pH reading crossed a threshold, a notification was sent to the user's email address. The complete experimental setup is demonstrated in FIG. 17.

The microneedle sensors were also tested for reproducibility. Three identical sensors were tested with the same sap solutions spiked with varying pH levels. The sensors showed highly reproducible pH measurements with a coefficient of variance of <2% (FIG. 18B).

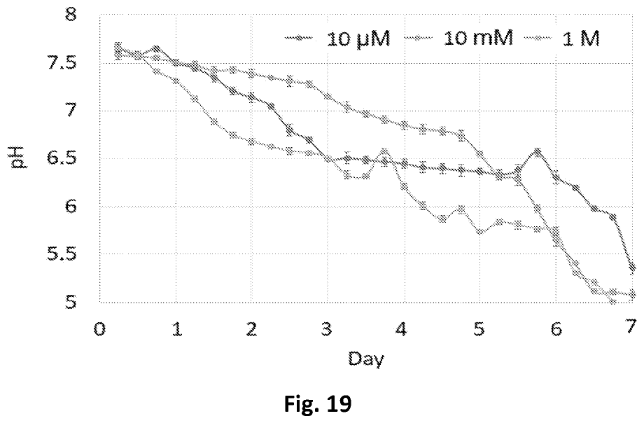

The leaf pH monitoring system was tested in live cabbage plants under different levels of salinity stresses. Three different concentrations, i.e., 10 μM, 10 μmM, and 1M of 20 μmL NaCl solutions were added to the soil and the sap pH levels were measured over 7 days. Measurements were taken four times a day, at 9 AM, 12 PM, 4 PM, and 8 PM. The results are shown in FIG. 19. The sap pH values decreased in response to the antioxidative defense response in the plant. The results show that the plant sensor was capable of monitoring the pH levels in response to salinity stress in real-time, thereby extending its application to remote monitoring of plant health.

### Example 15

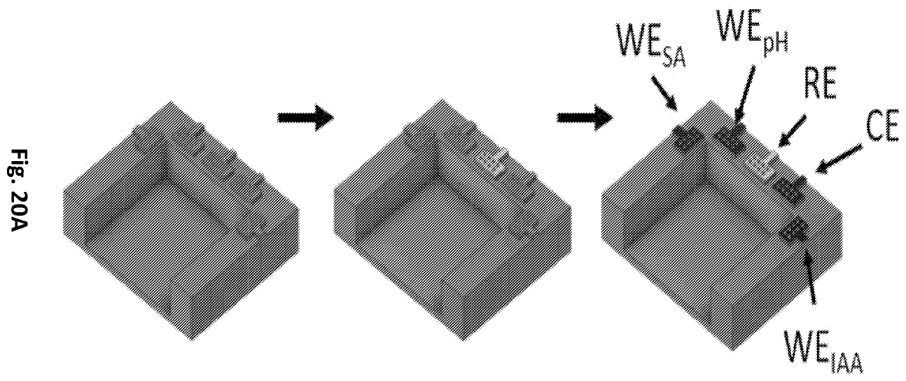

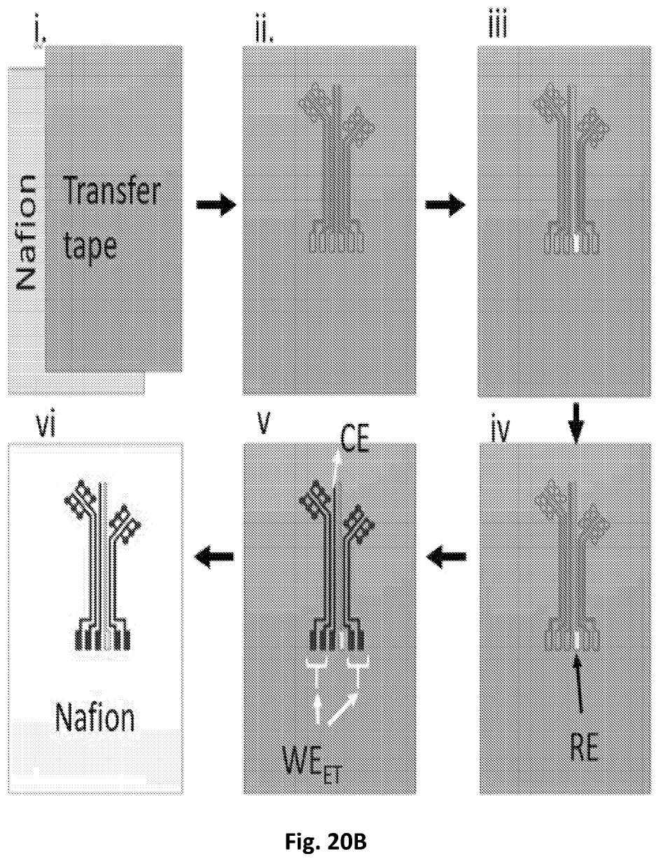

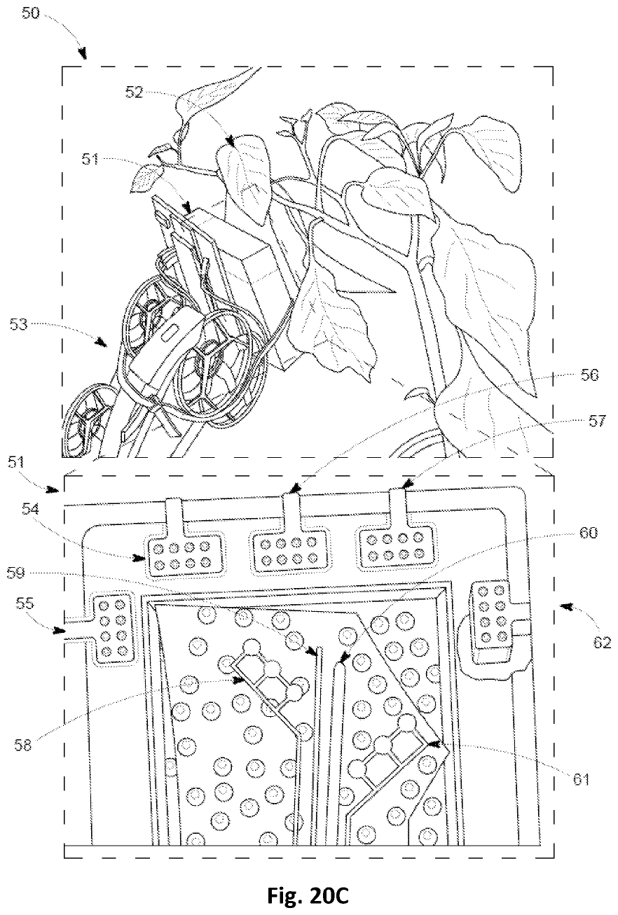

Electrode fabrication for a sensor suite. A sensor suite comprising five microneedle arrays was fabricated as follows. One array worked as the shared reference electrode (RE), one as the shared counter electrode (CE), and the other three arrays served as working electrodes for SA, IAA and pH sensors (WE_{SA}, WE_{IAA}, and WE_{pH}, respectively). To prepare the sensor suite, a 4 cm×3 cm×0.8 cm box was designed with an open ceiling and three sidewalls, as depicted in FIG. 20A. The ethylene sensor was placed inside a chamber enclosed by the three sidewalls, whereas the microneedle sensors were laid on top of the sidewalls. Each microneedle array consisted of eight pyramid-shaped microneedles, each having a square base of 800 μm, a height of 800 μm, and a tip angle of 60°. A Form 3B stereolithography printer was used to print the 3D box with the microneedles. BioMed Clear resin was used as the printing material to ensure biocompatibility of the microneedles.

The ethylene sensor was fabricated by a screen printing process, as shown in FIG. 20B. A thin Nafion sheet was used as the substrate material because it works as a solid-state electrolyte. To prepare the ethylene sensor, Nafion was covered with a transfer tape that worked as the stencil mask (i). Electrode patterns cut by a benchtop cutter (ii) and transfer tape from the reference electrode region was removed (iii). The reference electrode was printed with Ag/AgCl paste (iv). The working and counter electrode areas were exposed and printed with graphene ink (v), resulting in a dual working electrode for ethylene (WE_{ET}). As a result, the ethylene sensitivity was increased to sub-ppm levels. The electrodes were cured at 80° C. for 60 μminutes. The transfer tape was then removed resulting in electrodes transferred to the Nafion sheet (vi).

Synthesis of SA and IAA Selective Coatings. The working electrode of the SA sensor (WE_{SA}) was functionalized by a copper metal-organic framework (CuMOF)/nafion/carbon black nanocomposite, as described above. 4 μL of the nanocomposite solution was drop cast on WE_{SA} and dried at room temperature.

The IAA working electrode (WE_{IAA}) was modified with gold nanoparticles decorated graphene hydrogel nanocomposite. Briefly, 1 μmg mL⁻¹ of graphene oxide suspension was prepared. 20 μmL of this suspension was added to 2 μmL of 0.8511 μmg mL⁻¹ chloroauric acid solution and 1 μmL of triethylenetetramine. The resulting mixture was sonicated for 10 μminutes and then heated at 140° C. for 12 h. The obtained AuNP-GHs were cooled down to room temperature and then freeze-dried for 24 h to form powders that were stored in a desiccator. 4 μL of the as-prepared composite solution was drop cast on WE_{IAA} to form an electrode.

Synthesis of Ethylene Selective Coating. WE_{ET} was functionalized with a composite copper complex (I)-single-walled carbon nanotube coating for selective measurement of ethylene gas. All operations were carried out under an atmosphere of purified nitrogen and all solutions were prepared in deionized water. To prepare Na [HB(3,5-(CF₃)₂-pz)₃]), 0.40 g (10.6 μmmol) of NaBH₄ and 7.55 g (37 μmmol) of 3,5-(CF₃)₂-pz were mixed in just as much kerosene as was needed to form a homogeneous mixture. The mixture was slowly heated to 180-190° C. and kept for 4 hr at 190° C. The flux was partially submerged in silicone oil during the heating process. The solution was occasionally (every 15 μminutes) heated with a heat gun until pyrazole melted. During this period, a white solid slowly precipitated. After the mixture was cooled to room temperature, the resulting white solid was collected by suction filtration in air. It was washed several times with petroleum benzene and sucked dry in air to obtain Na [HB(3,5-(CF₃)₂-pz)₃]) as a white solid. Next, in order to form the Cu complex-1 coating, 8 μmg of [CF₃ SO₃Cu]₂_C₆H₆ were dissolved in 3 μmL dry, degassed toluene. Finally, 17 μmg of the freshly prepared Na[HB(3,5-(CF₃)₂-pz)₃]) were added to the mixture and stirred for 20 hrs at room temperature. The reaction mixture was filtrated through a Whatman 0.02 μm syringe filter and a colorless solution of Cu complex-1 with a concentration of ˜6 μmol/mL (6 μmM) was obtained. The prepared solution was stored at 4° C. in a refrigerator until further use. In a separate tube, 0.5 μmg of single-walled carbon nanotube (SWCNT) was added to a mixture of 0.8 μmL 1,2-dichlorobenzene and 1.16 μmL toluene, and the resulting mixture was sonicated for 2 hours to prepare a homogeneous solution. Next, the freshly prepared copper complex-1 solution was added to this mixture and sonicated for another 1 hour. Finally, 30 μL of this solution was drop cast on the working electrode (WE_{ET}) of the ethylene sensor.

Synthesis of pH Selective Coating. WE_{pH} was modified with polyaniline (PANI) nanofibers via electrodeposition. Polyaniline (PANI) nanofibers were deposited onto the graphene WE, as PANI-based electrode is highly sensitive to H₃O⁺ ions. In addition, the redox equilibrium between the H₃O⁺ and PANI provides high surface area, potential stability, biocompatibility, and reproducible performance. The PANI coating was deposited on the WE via electropolymerization method. The pH sensor electrodes were immersed in a mixture of 0.1 μM aniline and 0.1M HCl followed by 85 cycles of cyclic voltammetry (CV) for −0.2V to 0.6V at a 50 μmV/s scan rate.

### Example 16

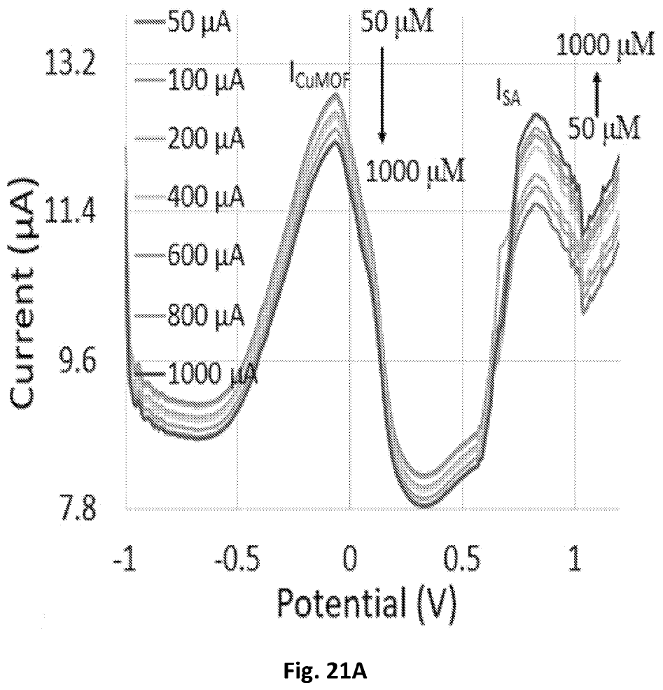

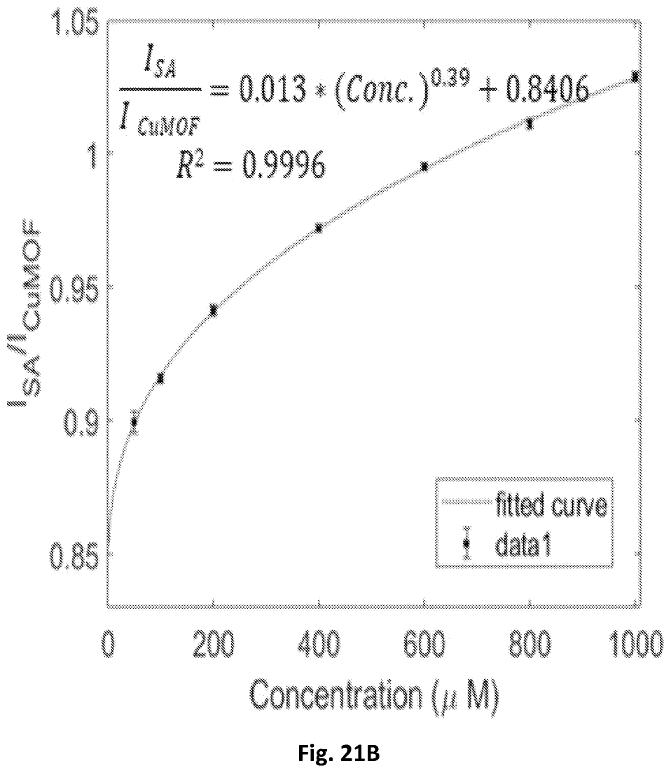

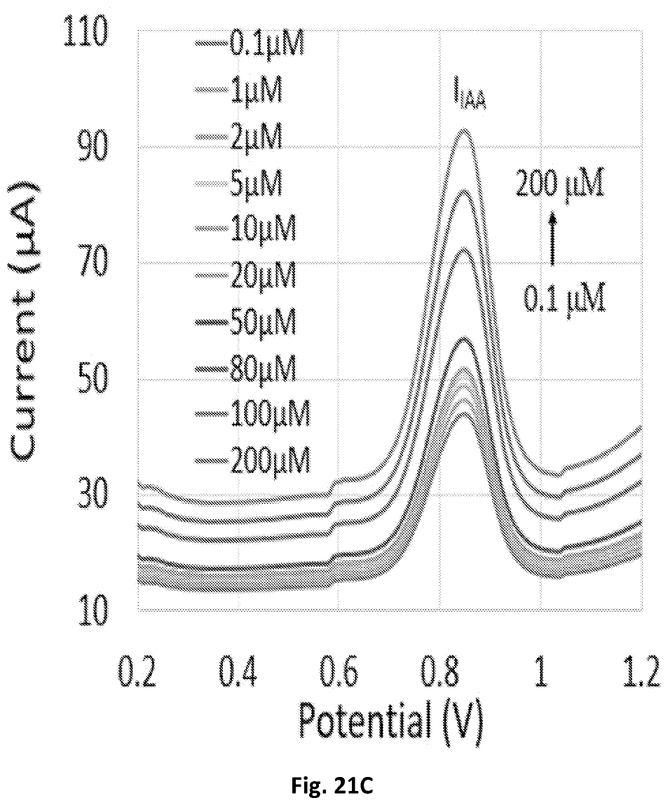

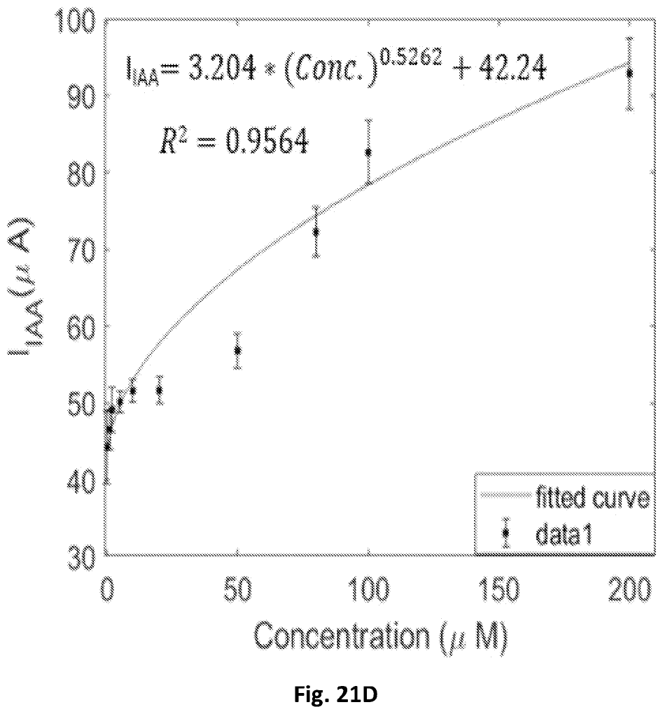

Electrochemical Detection of SA, IAA, and Ethylene. Electrochemical measurements were performed using differential pulse voltammetry (DPV) in a potential range from −1.0V to 1.2V for SA and from 0.2V to 1.2V for IAA (FIG. 21A and FIG. 21C). The SA sensor was calibrated for SA levels ranging from 50 μM to 1000 μM (FIG. 21B), while the IAA sensor was calibrated for IAA levels varying from 0.1 μM to 200 μM (FIG. 21D), commensurate with the typical SA and IAA concentrations found in plants. A ratiometric approach was used to calibrate the SA sensor, wherein the ratio of SA and CuMOF redox current peaks (I_{SA}/I_{CuMOF}) was plotted as a function of SA concentration and a power series curve was fitted to the data points (FIG. 21B). The SA and IAA sensors exhibited sensitivities of 0.005 μM⁻¹ and 0.8325 μA M⁻¹, with detection down to 0.93 μM and 0.08 μM, respectively.

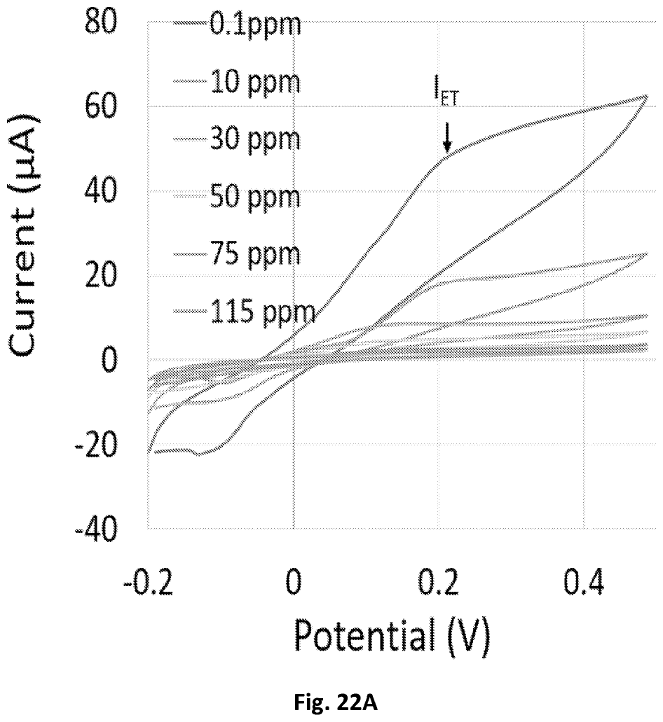

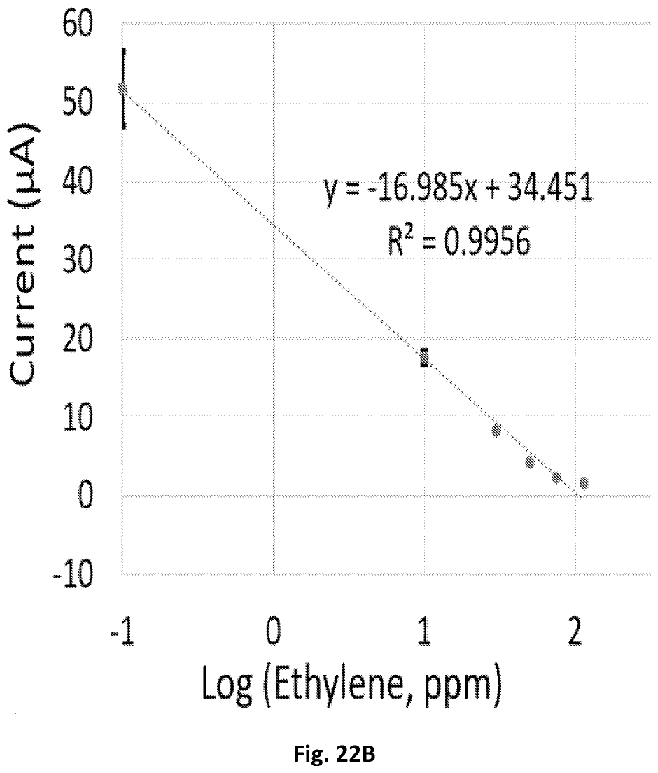

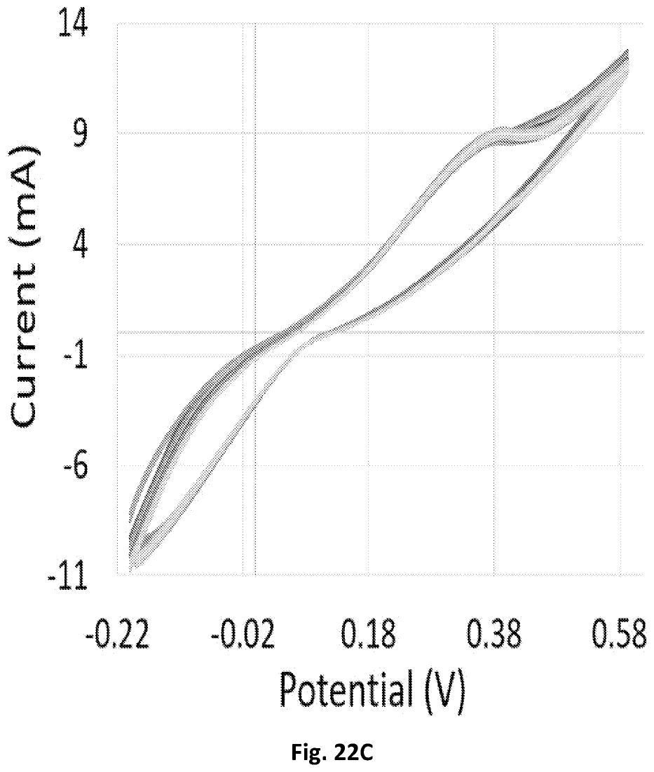

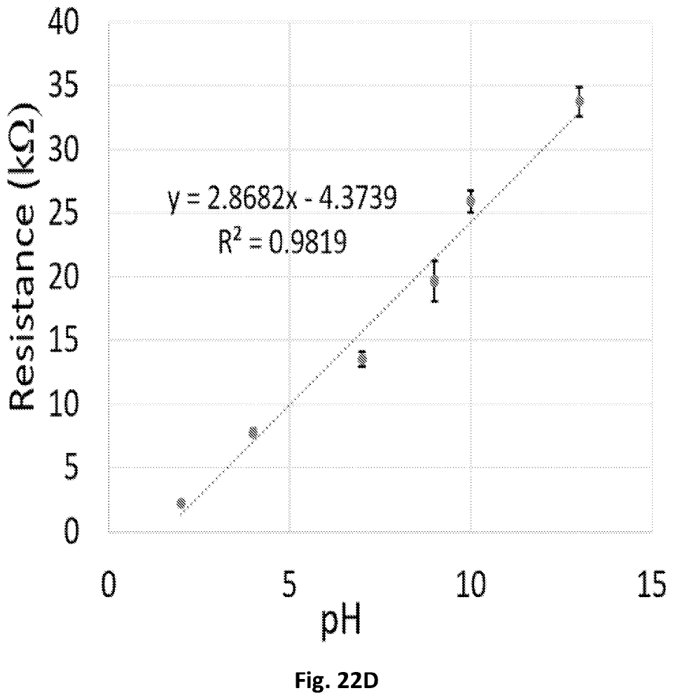

Cyclic Voltammetry (CV) method was used to conduct electrochemical characterization of the ethylene sensor. Different concentrations of ethylene gas were generated by controlling the gas flow rate and time in a flow chamber. The concentrations ranging from 0.1 ppm to 115 ppm were used to calibrate the ethylene sensor (FIG. 22A). The CV responses depict that the ethylene oxidation peak current (I_{ET}) lies between 0.12V and 0.17V. Upon exposure to a higher concentration of ethylene, the oxidation peak current decreased because ethylene molecules blocked the active sites in the carbon nanotube coating (FIG. 22A-22B).

pH Sensor Characterization. The pH sensor was calibrated with plant sap. The sap pH was varied by adding 0.1 μM HCl and 0.01M NaOH. CV responses for PANI deposition are shown in FIG. 22C. The pH sensor demonstrated an increase in the resistance measured across the electrodes with increasing pH value, as is illustrated in FIG. 22D.

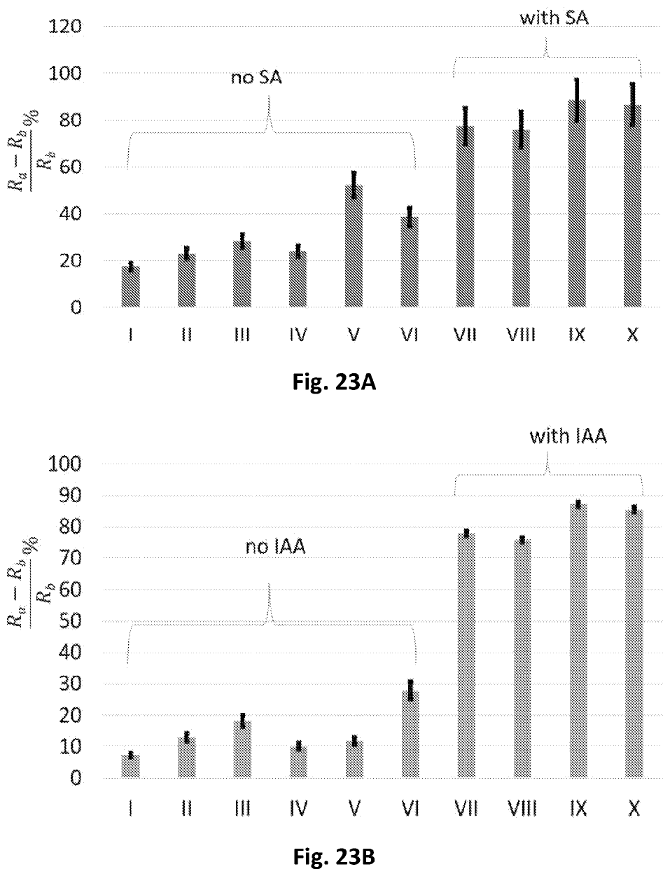

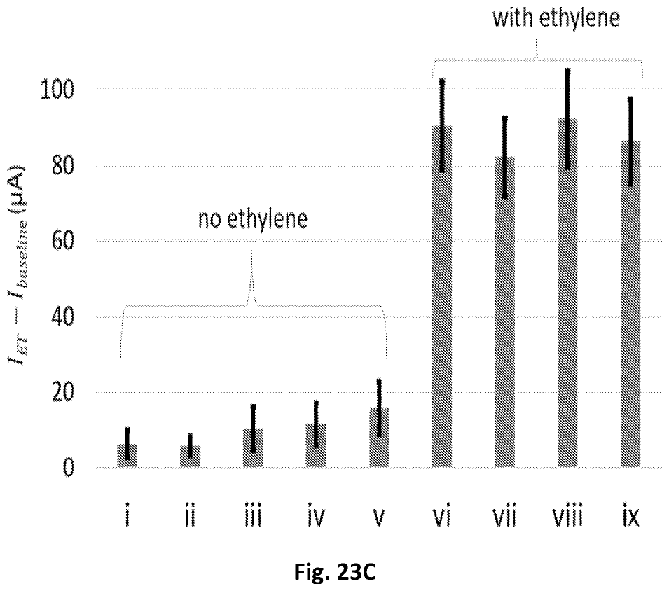

Selectivity Test for SA and IAA. The SA and IAA sensors were tested against several interfering species typically found in fruits/vegetables. Specifically, both SA and IAA sensors were tested under the following conditions shown in FIGS. 23A-23B: (i) Jasmonic acid (JA)=50 μM, (ii) L-Cysteine (L-Cys)=50 μM, (iii) glucose=50 μM, (iv) citric acid=50 μM, (v) ascorbic acid=50 μM, (vi) a mixture of JA, L-Cys, glucose, citric acid, and ascorbic acid (50 μM each), (vii) target hormone (SA/IAA)=100 μM, (viii) a mixture of ascorbic acid, JA, L-Cys, glucose, citric acid, ascorbic acid (50 μM each), and target hormone=100 μM, (ix) target (SA=900 μM or IAA=200 μM), (x) a mixture of ascorbic acid, JA, L-Cys, glucose, citric acid, ascorbic acid (50 μM each), and target (SA=900 μM or IAA=200 μM). In the absence of either SA or IAA (conditions (i) to (vi)), negligible responses were observed. However, when SA or IAA were present, respectively (conditions (vii) to (x)), a significant response was detected from each sensor. Rₐ=I_{SA}/I_{CuMOF} for SA sensor and I_{IAA} for IAA sensor, R_{b}=I_{baseline}/I_{CuMOF} for SA sensor and I_{baseline} for IAA sensor.

Selectivity Test for Ethylene. The ethylene sensor was tested against some common interfering gases emitted in an agricultural field. Specifically, ethylene sensor was tested under the following conditions shown in FIG. 23C:: (i) 50 ppm N₂, (ii) 50 ppm CH₄, (iii) 50 ppm N₂O, (iv) 50 ppm NH₃, (v) a mixture of 50 ppm of N₂, CH₄, N₂O, NH₃ each, (vi) 10 ppm ethylene, (vii) a mixture of 50 ppm N₂, CH₄, N₂O, NH₃ each and 10 ppm ethylene, (viii) 115 ppm ethylene and (ix) a mixture of 50 ppm N₂, CH₄, N₂O, NH₃ each and 115 ppm ethylene. In the absence of ethylene (conditions (i) to (v)), negligible responses were observed. However, when ethylene was present (conditions (vi) to (ix)), a significant response was detected.

Thus, as shown in FIGS. 23A-23C, all three sensors exhibited negligible responses in the absence of the target analyte, thereby confirming excellent selectivity.

pH Correction of SA and IAA. The SA and IAA values measured with the above described sensors were corrected for pH variations in bell pepper at different stages of ripening. The pH correction was performed using the equations below. The pH value of 7 was considered as a reference.

$${f}_{intercept ( pH )} = \frac{intercept ( pH )}{intercept ( pH = 7 )} \qquad ( 4 )$$

$${f}_{slope ( pH )} = \frac{slope ( pH )}{slope ( pH = 7 )} \qquad ( 5 )$$

$${f}_{exponent ( pH )} = \frac{exponent ( pH )}{exponent ( pH = 7 )} \qquad ( 6 )$$

The corrected intercept, slope, and exponent were found using the values computed in Equations (4-6):

$$intercept ( corrected ) = {f}_{intercept} ( pH ) * intercept ( initial ) \qquad ( 7 )$$

$$slope ( corrected ) = {f}_{slope} ( pH ) * slope ( initial ) \qquad ( 8 )$$

$$exponent ( corrected ) = {f}_{exponent} ( pH ) * exponent ( initial ) \qquad ( 9 )$$

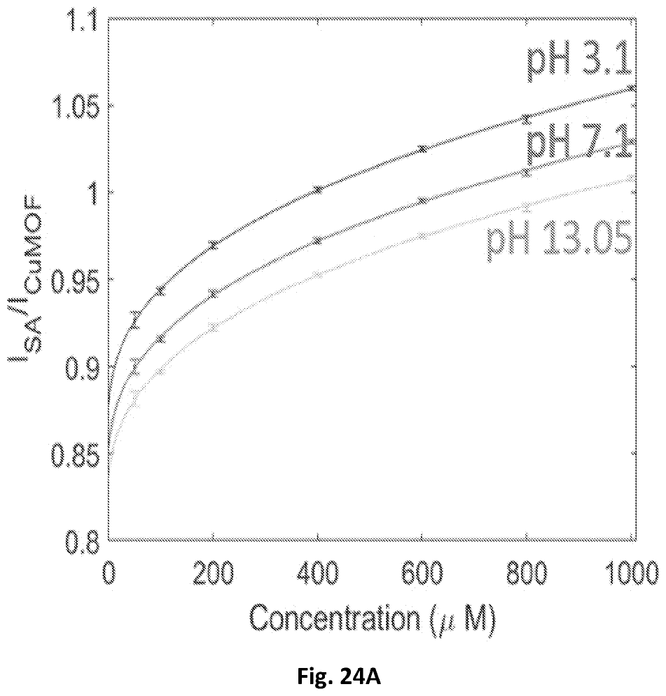

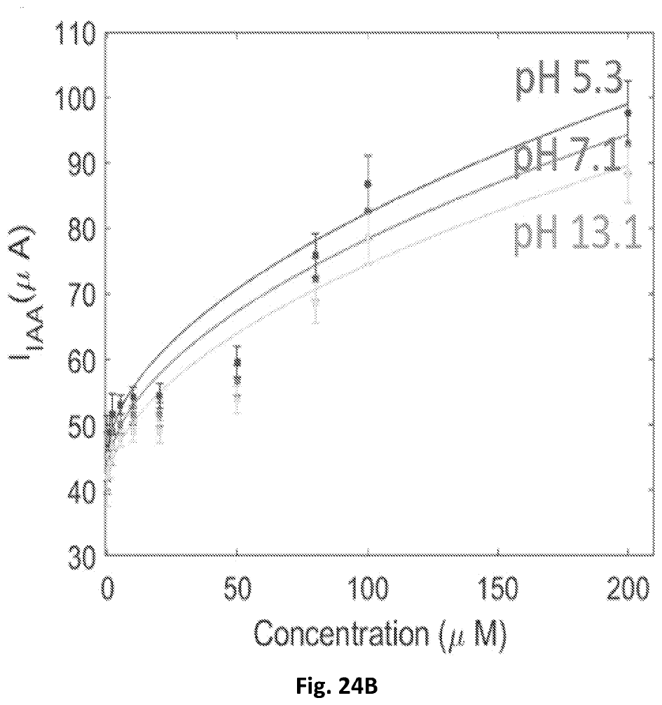

The calibration curves of SA and IAA sensors at different pH values are illustrated in FIGS. 24A-24B.

### Example 17

Real-time Monitoring of Fruit Ripening. The sensor suite, e.g., as shown in an exemplary sensor suite depicted in FIG. 20C, was deployed on bell peppers through a drone. As shown in FIG. 20C, the drone-interfaced plant sensor 50 comprising a sensor suite 51 was attached to the plant leaf 52 and configured to interface with a drone device 53. The sensor suite, as illustrated in 51, was used for multiplexed detection of ethylene, SA, and IAA levels with pH correction on the single platform. As illustrated in FIG. 20C, the sensor suite comprised a working electrode for pH (WE_{pH}) 54, a working electrode for SA (WE_{SA}) 55, a first reference electrode (RE) 56, a first counter electrode (CE) 57, a working electrode for ethylene (WE_{ET}) 58 and 61, a second CE 59, a second RE 60, and a working electrode for IAA (WE_{IAA}) 62.

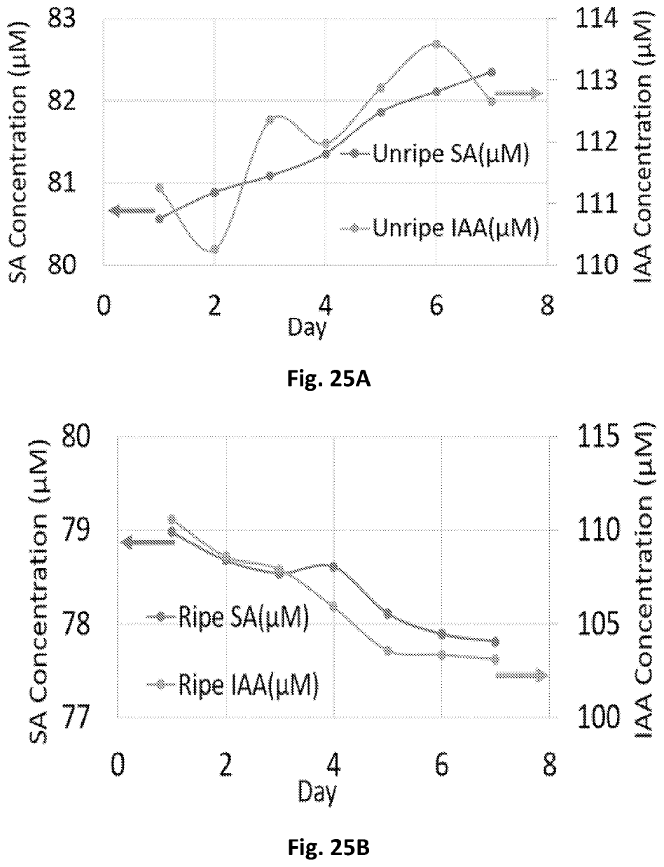

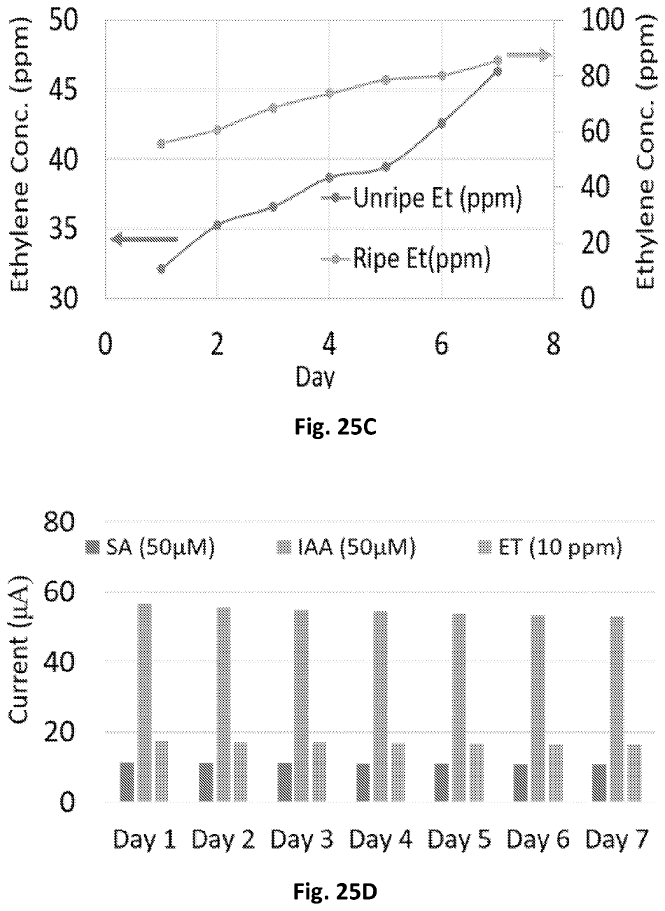

The SA, IAA, and ethylene levels were measured once a day for 7 consecutive days (FIGS. 25A-25C). The results show that both SA and IAA levels increased over time in unripe bell pepper, while the levels started to decrease once the bell pepper reached its maturity. The results are consistent with previous metabolic profiling studies (A. Oikawa, T. Otsuka, R. Nakabayashi, Y. Jikumaru, K. Isuzugawa, H. Murayama, K. Saito, K. Shiratake, “Metabolic profiling of developing pear fruits reveals dynamic variation in primary and secondary metabolites, including plant hormones,” PLoS One, vol. 10, pp. e0131408, 2015. doi: 10.1371/journal.pone.0131408), confirming the novel sensors described herein provide measurements that are consistent with conventional techniques using, for example, CE-TOF MS and LC-QTOF-MS, while, in contrast, being both easily deployable and scalable and providing real-time and continuous assessment of stress responses in plants.

Although the ethylene level showed a rising trend in both ripe and unripe bell peppers, the rate of change was higher in the unripe pepper. Stability analysis of SA, IAA, and ET sensors over one week. Peak current value decreased by 1.15%, 1.33%, and 2.5% for SA, IAA, and ET sensors, respectively (FIG. 25D). Thus, each of the sensors showed excellent stability over the 7 day measurement period.

As described above, the plant sensor was capable of monitoring the varying trend of hormone levels in ripe and unripe bell peppers.

### OTHER EMBODIMENTS

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

Other aspects, advantages, and modifications are within the scope of the following claims.

1. A plant sensor, comprising:
a biocompatible polymer substrate; and
one or more sensors disposed on the substrate, wherein each of the one or more sensors comprises a plurality of microneedles,
wherein:
a) the microneedles have a height dimension of 100 μm to 5,000 μm and a base-width dimension of 100 μm to 5,000 μm;
b) the microneedles have a vertex angle of 3° to 90°;
c) the microneedles have a bending angle of less than 15° at a pressure of 600 kPa;
d) the microneedles have a height dimension of 100 μm to 5,000 μm and a base-width dimension of 100 μm to 5,000 μm and the microneedles have a vertex angle of 3° to 90°;
e) the microneedles have a height dimension of 100 μm to 5,000 μm and a base-width dimension of 100 μm to 5,000 μm and the microneedles have a bending angle of less than 150 at a pressure of 600 kPa;
f) the microneedles have a vertex angle of 3° to 900 and the microneedles have a bending angle of less than 150 at a pressure of 600 kPa; or
g) the microneedles have a height dimension of 100 μm to 5,000 μm and a base-width dimension of 100 μm to 5,000 μm, the microneedles have a vertex angle of 3° to 90°, and the microneedles have a bending angle of less than 150 at a pressure of 600 kPa.

2. The plant sensor of claim 1, wherein the microneedles have a bending angle of 100 or less at a pressure of 200 kPa.

3. (canceled)

4. The plant sensor of claim 1, wherein a) at least one sensor is configured to measure a physical parameter and/or at least one sensor is configured to measure a chemical parameter, or b) the sensors are each independently configured to detect humidity, temperature, stem and/or leaf growth, pH, one or more phytohormones, or one or more volatile organic compounds, c) or both a) and b).

5. (canceled)

6. The plant sensor of claim 1, wherein at least one sensor is configured to detect Salicylic acid (SA), jasmonic acid (JA), abscisic acid (ABA), or indole-3-acetic acid (IAA).

7. (canceled)

8. The plant sensor of claim 1, wherein a) the microneedles have a height dimension of 200 μm to 4,000 μm, or 300 μm to 3,000 μm, or 400 μm to 2,000 μm, or 500 μm to 1,000 μm, or 600 μm to 800 μm, or b) the microneedles have a base-width dimension of 200 μm to 4,000 μm, or 300 μm to 3,000 μm, or 400 μm to 2,000 μm, or 500 μm to 1,000 μm, or 600 μm to 800 μm, c) or both a) and b).

9. (canceled)

10. The plant sensor of claim 1, wherein the microneedles have a vertex angle of 20° to 50°, or 30° to 40°.

11. (canceled)

12. The plant sensor of claim 1, wherein the plant sensor comprises a pH sensor integrated therein and is configured to perform pH correction of measured salicylic acid (SA) levels.

13. The plant sensor of claim 1, further comprising a data acquisition system, wherein the data acquisition system comprises a processor; a communication unit; and a power supply unit, and wherein the data acquisition system is in communication with the one or more sensors.

14. The plant sensor of claim 13, further comprising a potentiostat in communication with the one or more sensors, and wherein the potentiostat is in communication with one or more of the processor, the communication unit, the power supply unit, and the data acquisition system.

15-41. (canceled)

42. A method for continuously measuring one or more phytohormones in a plant, comprising:
a) attaching to the plant:
i) a reference electrode (RE);
ii) a counter electrode (CE); and
iii) one or more working electrode (WE) configured to detect a phytohormone,
b) wherein each electrode comprises a plurality of microneedles, and
c) wherein each electrode is operatively connected to an electrode control unit;
d) applying a potential corresponding to a peak current for the one or more phytohormones;
e) measuring at least one signal correction parameter;
determining the concentration of the one or more phytohormones based on the peak current using a pre-determined calibration plot, wherein the pre-determined calibration plot is based on the measured value of the at least one signal correction parameter.

43. The method of claim 42, wherein the phytohormone is selected from Salicylic acid (SA), jasmonic acid (JA), abscisic acid (ABA), or indole-3-acetic acid (IAA).

44. (canceled)

45. The method of claim 42, wherein the at least one signal correction parameter is selected from temperature, humidity, pH, and an analyte.

46. The method of claim 45, wherein the analyte is a second phytohormone.

47. The method of claim 42, wherein the electrode control unit comprises at least one of a potentiostat and a data acquisition system.

48. (canceled)

49. (canceled)

50. The method of claim 42, wherein the electrode control unit comprises a processor and a communication unit, and wherein the electrode control unit comprises a non-transitory computer-readable medium communicatively coupled to a processor, the non-transitory computer-readable medium having stored thereon computer software comprising a set of instructions that, when executed by the processor, causes the electrode control unit to send the electrode data from the electrodes-is-sent, via the communication unit, to an Internet of Things (IoT) cloud server configured to interact with one or more IoT-capable devices.

51. (canceled)

52. (canceled)

53. The method of claim 50, wherein the instructions are configured to perform a signal calibration.

54. The method of claim 53, wherein the calibration comprises at least one of a pH-based signal correction, a temperature-based signal correction, a humidity-based signal correction, or a signal calibration based on the signal of an analyte.

55. The method of claim 50, wherein the electrodes are attached to a leaf of the plant or to a stem of a plant.

56. (canceled)

57. The method of claim 50, wherein the electrodes are attached to at least two locations of the same plant.

58. The method of claim 57, further comprising measuring kinetics and/or distribution of the one or more phytohormones in the plant.

59. (canceled)

60. (canceled)

61. (canceled)

62. The method of claim 50, further comprising modifying and/or applying a pesticide treatment in response to the concentration of the one or more phytohormones.

63. The method of claim 50, further comprising modifying and/or applying a fertilizer or nutrient treatment in response to the concentration of the one or more phytohormones.

64. The method of claim 50, further comprising harvesting the plant in response to the concentration of the one or more phytohormones.

65. (canceled)

66. (canceled)

67. The method of claim 50, wherein the one or more phytohormones are detected with a deviation of less than 10%, or less than 5%, or less than 1%, or less than 0.5%, or less than 0.1%, wherein the deviation is across at least three repeated measurements.

68. (canceled)

69. A plant sensor, comprising:
a) a biocompatible polymeric substrate;
b) two or more electrodes disposed on the substrate, wherein each electrode comprises a plurality of microneedles;
c) a power supply unit;
d) an electrode control unit; and
e) a voltage booster connected to the two or more electrodes;
f) wherein the microneedles have a height dimension of 100 μm to 5,000 μm and a base-width dimension of 100 μm to 5,000 μm and the microneedles have a vertex angle of 3° to 90°; and
g) wherein the power supply unit and the electrode control unit are in communication with the two or more electrodes.

70. The plant sensor of claim 69, comprising a reference electrode (RE), at least one working electrode (WE), and optionally a counter electrode (CE).

71. The plant sensor of claim 69, wherein the plurality of microneedles are coated with a coating selected from a graphene ink, an Ag/AgCl paste, a metal organic framework (MOF), a graphene hydrogel nanocomposite, a poly(3,4-ethylenedioxythiophene) polystyrene sulfonate (PEDOT:PSS) cross-linked with 3-glycidyloxypropyl)trimethoxysilane (GOPS), a polyaniline (PANI) based nanofiber, or a combination thereof.

72. The plant sensor of claim 69, wherein the RE is coated with Ag/AgCl paste.

73. The plant sensor of claim 69, wherein the WE is coated with a PANI.

74. The plant sensor of claim 69, wherein the electrode control unit comprises a non-transitory computer-readable medium communicatively coupled to a processor, the non-transitory computer-readable medium having stored thereon computer software comprising a set of instructions that, when executed by the processor, causes the electrode control unit to:
a) receive electrode data from each of the two or more electrodes; and
b) send, via the communication unit, the sensor data to an external device.

75. (canceled)

76. (canceled)

77. (canceled)

78. (canceled)

79. (canceled)

80. (canceled)

81. (canceled)

82. (canceled)

83. (canceled)

84. A method for continuously measuring pH in a plant, comprising:
a) attaching to the plant:
i) a reference electrode (RE);
ii) a working electrode (WE) configured to detect ions,
b) wherein each electrode comprises a plurality of microneedles, and
c) wherein each electrode is operatively connected to a voltage booster and an electrode control unit;
d) measuring an output voltage;
e) determining the pH based on a pre-determined calibration plot.

85. The method of claim 84, wherein the RE is coated with Ag/AgCl paste.

86. The method of claim 84, wherein the WE is coated with PANI.

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. A biocompatible polymer plant sensor, comprising:
a substrate;
at least three sidewalls each attached to the substrate on a first side; and
a chamber;
wherein the chamber is enclosed by the at least three sidewalls and the substrate,
one or more sensors comprising a plurality of microneedles disposed on a second side of at least one sidewall, the second side being opposite to the first side attached to the substrate, and
one or more sensors disposed in the chamber,
wherein:
a) the microneedles have a height dimension of 100 μm to 5,000 μm and a base-width dimension of 100 μm to 5,000 μm;
b) the microneedles have a vertex angle of 3° to 90°;
c) the microneedles have a bending angle of less than 15° at a pressure of 600 kPa;
d) the microneedles have a height dimension of 100 μm to 5,000 μm and a base-width dimension of 100 μm to 5,000 μm and the microneedles have a vertex angle of 3° to 90°;
e) the microneedles have a height dimension of 100 μm to 5,000 μm and a base-width dimension of 100 μm to 5,000 μm and the microneedles have a bending angle of less than 150 at a pressure of 600 kPa;
f) the microneedles have a vertex angle of 3° to 900 and the microneedles have a bending angle of less than 150 at a pressure of 600 kPa; or
g) the microneedles have a height dimension of 100 μm to 5,000 μm and a base-width dimension of 100 μm to 5,000 μm, the microneedles have a vertex angle of 3° to 90°, and the microneedles have a bending angle of less than 150 at a pressure of 600 kPa.

103. The biocompatible polymer plant sensor of claim 102, wherein the chamber is open to the surroundings on at least one side.

104. The biocompatible polymer plant sensor of claim 102, further comprising a fourth sidewall.

105. The biocompatible polymer plant sensor of claim 102, wherein three sidewalls have one or more sensors comprising a plurality of microneedles.

106. The biocompatible polymer plant sensor of claim 102, wherein the biocompatible polymer plant sensor is configured to interface with a drone.

107. The biocompatible polymer plant sensor of claim 102, wherein the one or more sensors disposed in the chamber are screen printed electrodes.

108. The biocompatible polymer plant sensor of claim 102, wherein the screen printed electrodes comprise a reference electrode (RE), a counter electrode (CE), and at least one working electrode (WE).

109. The biocompatible polymer plant sensor of 108, wherein the working electrode is a dual working electrode or wherein the working electrode comprises an ethylene sensor.

110. (canceled)

111. The biocompatible polymer plant sensor of claim 102, wherein the selectivity for target analytes is at least 1.1× higher than one or more interfering species, or wherein a peak current value detected has a decrease of 2.5% or less over at least seven days.

112. (canceled)

113. (canceled)

114. (canceled)

115. (canceled)

116. (canceled)
